(19) United States
(12) Patent Application Publication
(10) Pub. No.: US 2023/0364608 A1
(43) Pub. Date: Nov. 16, 2023

(54) MEMBRANE INLET FOR CHEMICAL ANALYSIS WITH CONTINUOUS FLOW SAMPLE DEGASSING

(71) Applicant: Ryan Bell, Lafayette, CO (US)

(72) Inventor: Ryan Bell, Lafayette, CO (US)

(21) Appl. No.: 18/317,906

(22) Filed: May 15, 2023

Related U.S. Application Data
(60) Provisional application No. 63/341,932, filed on May 13, 2022.

(51) Int. Cl. B01L 3/00

(52) U.S. Cl. CPC B01L 3/502723; B01L 2300/0663; B01L 2200/0684; B01L 2200/027; B01L 2400/0487

(57) ABSTRACT

A membrane inlet for chemical analysis with continuous flow sample degassing of at least two analytes within a sample solution is disclosed. The membrane inlet comprises: a housing having a sample volume and an analysis volume; a long membrane within the housing that physically separates the sample volume from the analysis volume; a sensor configured to measure a concentration for each of the analytes in the analysis volume; and a controller in signal communication with the sensor. The housing is configured to receive a flow of the sample solution through the sample volume and the long membrane is configured to permeate the at least two analytes from the sample solution into the analysis volume. Multiple inlets and long membranes may be interconnected in a series or parallel arrangement.

### CROSS-REFERENCE TO RELATED APPLICATIONS

The application claims priority under 35 U.S.C. § 119(e) to U.S. Provisional Patent Application No. 63/341,932, titled “Membrane Inlet For Chemical Analysis with Sample Degassing,” filed on May 13, 2022, which is herein incorporated by reference in its entirety.

### BACKGROUND

### 1. Field

The present disclosure relates in general to systems and methods for performing measurements for chemical analysis, and more specifically, to systems and methods for performing in situ measurements for chemical analysis.

### 2. Related Art

At present, measurements of dissolved gases by membrane separation are an important way of performing chemical analysis on liquids and gases having chemical atoms, or molecules (i.e., species) that need to have an analyte (i.e., a chemical species that is a substance or chemical constituent that is of interest in an analytical procedure) separated and measured from a bulk sample matrix (i.e., the components of the sample other than the analyte of interest).

As an example, the detection of dissolved gases in seawater plays an important role in oceanic observations and exploration and is essential for studying the ocean's environment and ecosystem. CO₂ is a key factor in global warming, and O₂ is an important sign of net biological oxygen production. There is a certain relationship between CO₂ and O₂ in primary production (photosynthesis and chemosynthesis) and secondary production (respiration). Dissolved H₂ can be a key parameter of thermodynamic equilibria and kinetic processes in water-rock interaction processes. Thus, there is significant scientific and environmental value in tracking the concentrations of these and other dissolved gases in the ocean. However, the low concentrations of dissolved gases and the complex oceanic environment are significant challenges for in situ dissolved gases sensors.

As another example, monitoring volatile compounds (i.e., substances capable of readily changing from a solid or liquid form to a vapor) in sewer systems is of high importance because of the toxic and corrosive nature of various nuisance chemicals that are generated in sewer systems such as, for example, hydrogen sulfide (H₂S). By monitoring and identifying the presence and location of any generated H₂S, targeted treatment can be applied to this location that eventually minimizes the use of chemicals and lowers the environmental effect within the sewer system.

Moreover, as another example, monitoring of volatile organic compounds (e.g., natural gas and other light hydrocarbons) dissolved in water bodies may be of important industrial and commercial interest with respect to the environmental monitoring of offshore oil and gas infrastructure and exploration of oil and gas resources.

A problem exists, however, when ratiometric measurement tools are utilized for in situ chemical analysis. In general, in situ means “in the reaction mixture” or “operations or procedures that are performed in place” and in the chemical field there are numerous situations in which chemical intermediates are synthesized in situ in various processes. This may be done because the species is unstable, and cannot be isolated, or simply out of convenience.

Examples of in situ chemical analysis include performing chemical analysis with sensitivity and specificity where the chemical species of interest needs to be separated from the bulk sample matrix (as an example, in sample pre-concentration). Moreover, many means of chemical analysis require that the chemical species be in a gas phase (needed e.g. for sample vaporization of the chemical species). A problem is that in situ and online (also known as continuous) chemical analysis procedures typically necessitate that the two steps of sample pre-concentration and vaporization be performed with limited or no sample preparation.

Current approaches to solve this problem include the utilization of a thin membrane within a chemical analyzer inlet (known as a membrane inlet) that extracts and volatizes (i.e., cause to evaporate or disperse in vapor) the gaseous or aqueous sample hydrophobic substances (i.e., substances that are composed of non-polar molecules that repel bodies of water and attract other neutral molecules and non-polar solvents) via pervaporation through the thin membrane. As such, membrane inlets are a popular choice for online and in situ analysis because membrane inlets achieve these goals by a simple means.

As an example, in FIG. 1, a system block diagram of an example of a known measurement system 100 having a membrane inlet 102 and an analyzer 104 is shown. The membrane inlet 102 may be physically connected to the analyzer 104 via a connecting fluidic tube 106. The membrane inlet 100 includes a cavity 108, a sample inlet 110, a sample outlet 112, membrane capillary 114, thermocouple 116, and volatile analyte 118 (such as permanent gases or volatile organic carbons VOCs). The membrane inlet 100 also includes a known membrane physical support 120 surrounding the membrane capillary 114. In this example, the membrane capillary 114 might be supported by the membrane's inherent strength, itself, or the membrane physical support 120, where the membrane physical support 120 may include wound wire, sintered materials, or perforated materials located in the interior cavity of the surface of the membrane.

The analyzer 104 may be, for example, a spectroscopy analyzer or mass spectrometer that utilizes mass spectrometry (MS) and may include a vacuum chamber 122 having an electron source 124, an accelerator section 126, deflection electromagnets 128, outlet 130 to a vacuum pump, and a detector 131.

MS is an analytical technique that is used to measure the mass-to-charge ratio (m/z) of ions. The results are presented as a mass spectrum, a plot of intensity as a function of the mass-to-charge ratio. MS is used in many different fields and is applied to pure samples as well as complex mixtures because MS is known to be a versatile and powerful chemical sensing technique.

Generally, in MS systems (also known as mass spectrometers), like analyzer 104, analytes are transported from their normal state (e.g., solid phase or solution) into the vacuum chamber 122 of the mass spectrometers through a sample interface. After entering the vacuum chamber 122, ionized analytes 132 are then dispersed according to their m/z by some combination of electrical and magnetic fields 134 produced by the electromagnets 128. The ion signal is recorded as a function of mass-to-charge ratio, typically using a high-gain electron multiplier or Faraday-cup detector (e.g., detector 126) and the measured intensities for each m/z result in the mass spectrum and can often be related to the concentration of the analyte in the original sample, or possibly be used for identification of unknowns in a complex mixture. Mass spectrometers are, therefore, utilized in many fields of science and engineering.

In this example, the membrane inlet 102 allows for continuous introduction of multiple volatile species 118 with no sample preparation. Moreover, the analyzer 104 utilizing MS allows for sensitive simultaneous detection of multiple chemical species with high specificity.

Unfortunately, for in situ ratiometric measurements of dissolved gases in dynamic environments, these measurement systems that utilize membrane inlets have a number of problems that make ratiometric measurements difficult and time consuming to perform because of the characteristics and operation of the membranes under high pressure, temperature variations, membrane conditions and calibration, and flow characteristics of different analytes through the membrane.

As an example, FIG. 2 is a system block diagram of a known membrane extractor 200 that performs membrane equilibration analysis. The membrane extractor 200 includes a housing 202, membrane 204, and transducer 206. The housing 202 includes sample volume 208, analysis volume 210, inlet feed 212 for a sample or calibration standard 214, and outlet 216 for rejected waste 218. The transducer 206 is located within an analysis volume 210 of the housing 202. The membrane 204 separates the sample volume 208 from the analysis volume 210, and analysis volume 210 has a fixed volume that is sealed by the membrane 204. In this example, the transducer 206 is a measurement device configured to measure the concentration of an analyte in the analysis volume 210.

In an example of operation, by connecting the membrane 204 to the sealed and fixed analysis volume 210 and providing enough time for analyte transport to achieve equilibrium across the membrane 204, an analysis (i.e., measurement with the transducer 206) that is independent of the membrane 204 permeability can be made. In general, utilizing this equilibration methodology can provide very accurate measurements of analyte concentrations (ratiometric or otherwise), but unfortunately this approach is limited by slow and asymptotic equilibration times. As an example, the time to reach equilibrium may vary from as little as 30 mins to days. However, in field environments, it may not even be possible to reach a steady state condition to take measurements, as the environment conditions may be continually shifting. Additionally, poor post-analysis recovery to instrumental baseline (regeneration) and limitations to non-destructive analysis techniques are additional issues that frequently limit this type of method.

Other known approaches include utilizing a steady state approach instead of an equilibrium approach so as to accelerate the time in obtaining the measurements of the analyte concentrations. In a steady state approach, the sample 214 is flowed through the sample volume 208 at constant rate and any permeated analytes in the analysis volume 210 are evacuated or otherwise removed from the analysis volume (e.g. sequestered, reacted, quenched). such that measurements signals produced by the transducer 206 will reach a steady state signal amplitude over time because as the membrane 204 is exposed with sample 214 long enough, the amount of analytes that permeate through the membrane 204 become constant.

While significantly more rapid, this analysis methodology can sometimes suffer from poor accuracy as the analyte permeation rate through the membrane 204 can vary based on a numerous complexities and the variability in membrane permeability causes imprecise measurements. Moreover, this technique is limited by the necessity to use external standards that must incorporate the combined effects of sorption into the membrane 204, diffusion through the membrane 204, desorption and, finally, the analysis technique. This can be a major problem if the membrane extractor 200 is utilized in situ in a hostile environment such as, for example, in the ocean at depth of, for example, more than 1,000 meters where conditions can change very quickly in terms of solution concentrations, pressure, and temperature because any calibration of the membrane extractor 200 must also be done in situ with calibration systems.

As such, there is a need for a system and method that solves these problems.

### SUMMARY

A membrane inlet for chemical analysis with continuous flow sample degassing of at least two analytes within a sample solution is disclosed. In an embodiment, the membrane inlet comprises: a housing having a sample volume and an analysis volume; a long and/or thin membrane within the housing that physically separates the sample volume from the analysis volume; a sensor configured to measure a concentration for each of the analytes in the analysis volume; and a controller in signal communication with the sensor. The housing is configured to receive a flow of the sample solution through the sample volume and the long and/or thin membrane is configured to permeate the at least two analytes from the sample solution into the analysis volume. The controller includes a memory, a machine-readable medium having executable instructions, and at least one processor in signal communication with the machine-readable medium, the at least one processor configured to perform operations based on the executable instructions. The operations include: producing a constant flow of the sample solution through the sample volume and over a surface of the long membrane; permeating the at least two analytes from the sample solution in the sample volume into the analysis volume as the sample solution passes along the length of the membrane until the sample solution in the sample volume has been approximately completely degassed prior to exiting an outlet of the sample volume; evacuating the analysis volume into a measurement chamber; producing a first measurement signal, with the sensor, corresponding to a first concentration of a first analyte having permeated through the membrane into the analysis volume; producing a second measurement signal, with the sensor, corresponding to a second concentration of a second analyte through the membrane into the analysis volume; and determining a ratiometric measurement for the first analyte and the second analyte based on the first measurement signal and the second measurement signal.

Alternatively, multiple interconnected inlet chambers may be fluidically connected with one another to achieve an equivalent long membrane. In such an embodiment having for example two chambers, the membrane inlet may comprise: a first housing having a first sample volume, a first analysis volume, and a first exhaust fluidically connected to the first sample volume, wherein the first housing is configured to receive a flow of the sample solution through the first sample volume and the first exhaust; a first long membrane within the first housing that physically separates the first sample volume from the first analysis volume, wherein the first long membrane is configured to permeate the at least two analytes from the sample solution into the first analysis volume; a second housing having a second sample volume, a second analysis volume, and a second exhaust, wherein the second housing is configured to receive the flow of the sample solution through the second sample volume, wherein the second analysis volume is fluidically connected to the first analysis volume to receive sample solution from the first exhaust; a second long membrane within the second housing that physically separates the second sample volume from the second analysis volume, wherein the second long membrane is configured to permeate the analytes from the sample solution into the second analysis volume; a sensor configured to measure a concentration for each of the analytes in the second analysis volume; and a controller in signal communication with the sensor. The controller includes a memory, a machine-readable medium having executable instructions, and at least one processor in signal communication with the machine-readable medium, the at least one processor configured to perform operations based on the executable instructions. The operations include: producing a constant flow of the sample solution through the first sample volume and over a first surface of the first long membrane; evacuating the first analysis volume into the second analysis volume; permeating the at least two analytes from the sample solution in the first sample volume into the first analysis volume until the sample solution in the first sample volume has been partially degassed prior to exiting the first outlet of the first sample volume; injecting the constant flow of the sample solution into and through the second sample volume and over a second surface of the second long membrane; permeating the at least two analytes from the sample solution in the second sample volume into the second analysis volume until the sample solution in the second sample volume has been approximately completely degassed prior to exiting the second outlet of the second sample volume; producing a first measurement signal, with the sensor, corresponding to a first concentration a first analyte through the first long membrane into the first analysis volume and the second long membrane into the second analysis volume; producing a second measurement signal, with the sensor, corresponding to a second concentration of a second analyte through the first long membrane into the first analysis volume and the second long membrane into the second analysis volume; and determining a ratiometric measurement for the first analyte and the second analyte based on the concentration of first analyte and the concentration of the second analyte.

Further disclosed is a method for chemical analysis with continuous flow sample degassing of a plurality of analytes within a first sample solution utilizing a first membrane inlet having a first housing, a first long membrane within the first housing, a second sample solution utilizing a second membrane inlet having a second housing, a second long membrane within the second housing, and a sensor, wherein the first housing has the first sample volume and a first analysis volume physically separated by the first long membrane, and the second housing has the second sample volume and a second analysis volume physically separated by the second long membrane, wherein the first housing and second housing are fluidically connected in parallel. The method comprises: producing a first constant flow of the sample solution through the first sample volume and over a first surface of the first long membrane; producing a second constant flow of the sample solution through the second sample volume and over a second surface of the second long membrane; permeating a first sub-plurality of analytes from the sample solution in the first sample volume into the first analysis volume until the sample solution in the first sample volume has been approximately completely degassed prior to exiting an first outlet of the first sample volume; permeating the second sub-plurality of analytes from the sample solution in the second sample volume into the second analysis volume until the sample solution in the second sample volume has been approximately completely degassed prior to exiting an second outlet of the second sample volume; evacuating the first analysis volume and second analysis volume; producing a first measurement signal, with the sensor, corresponding to a first concentration a first analyte through the first long membrane and second long membrane into the first analysis volume and second analysis volume; producing a second measurement signal, with the sensor, corresponding to a second concentration of a second analyte through the first long membrane and second long membrane into the first analysis volume and second analysis volume; and determining a ratiometric measurement for the first analyte and the second analyte based on the first measurement signal and the second measurement signal.

Other devices, apparatuses, systems, methods, features, and advantages of the invention will be or will become apparent to one with skill in the art upon examination of the following figures and detailed description. It is intended that all such additional devices, apparatuses, systems, methods, features, and advantages be included within this description, be within the scope of the invention, and be protected by the accompanying claims.

### BRIEF DESCRIPTION OF THE FIGURES

The invention may be better understood by referring to the following figures. The components in the figures are not necessarily to scale, emphasis instead being placed upon illustrating the principles of the invention. In the figures, like reference numerals designate corresponding parts throughout the different views.

FIG. 1 is a system block diagram of an example of a known approach for a membrane inlet.

FIG. 2 is a system block diagram of a known membrane extractor that performs membrane equilibration analysis.

FIG. 3 is a system block diagram of an example implementation of a membrane inlet configured for fixed volume analysis.

FIG. 4 is a plot of a measured signal corresponding to the measured permeation flux through the membrane in an accumulation method.

FIG. 5 is a plot of a measured signal corresponding to the measured permeation flux through the membrane in an integration method.

FIG. 6 is a flowchart of an example implementation of an accumulation method performed by the membrane inlet shown in FIG. 3.

FIG. 7 is a flowchart of an example implementation of an integration analysis method performed by the membrane inlet shown in FIG. 3.

FIG. 8 is a flowchart of an example implementation of a recirculation method performed by the membrane inlet shown in FIG. 3.

FIG. 9 is a system block diagram of an example of an implementation of a membrane inlet within a measurement system in accordance with the present disclosure.

FIG. 10 is a plot of a measured signal corresponding to the measured permeation flux through the membrane in another integration method in accordance with the present disclosure.

FIG. 11 is a system block diagram of another membrane inlet utilizing a long membrane with continuous sample flow, to approximately completely degas a sample solution in accordance with the present disclosure.

FIG. 12 is the system block diagram shown in FIG. 11, where the sample solution is partially degassed in accordance with the present disclosure.

FIG. 13 is the system block diagram shown in FIG. 12, where the sample solution is recirculated through the membrane inlet.

FIG. 14 is a system block diagram of a membrane inlet system utilizing multiple interconnected membrane inlets connected in series in accordance with the present disclosure.

FIG. 15 is a system block diagram of a membrane inlet system utilizing multiple interconnected membrane inlets connected in parallel in accordance with the present disclosure.

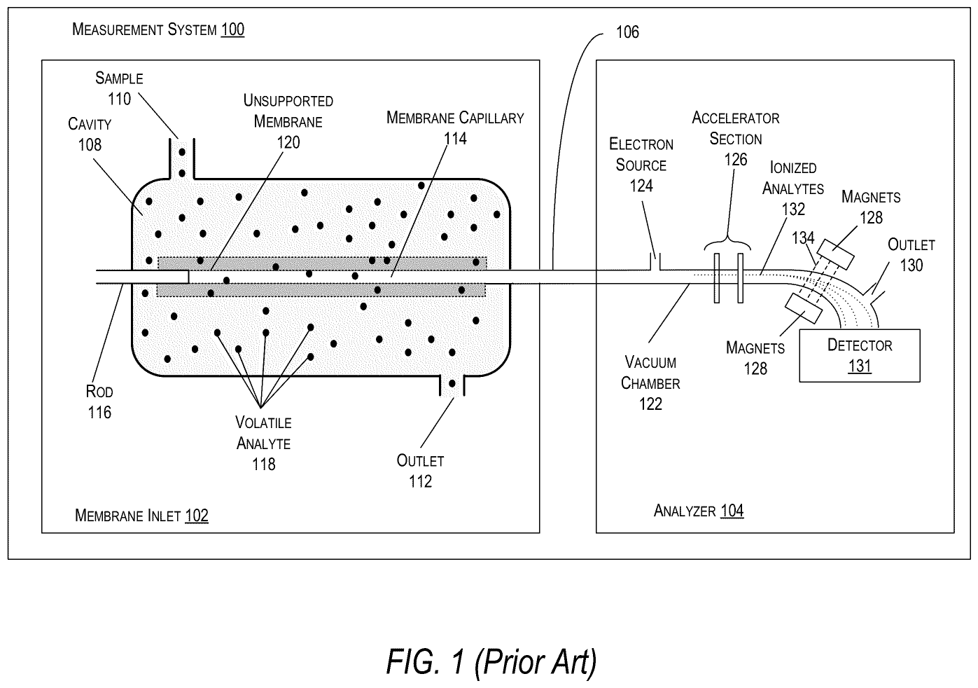

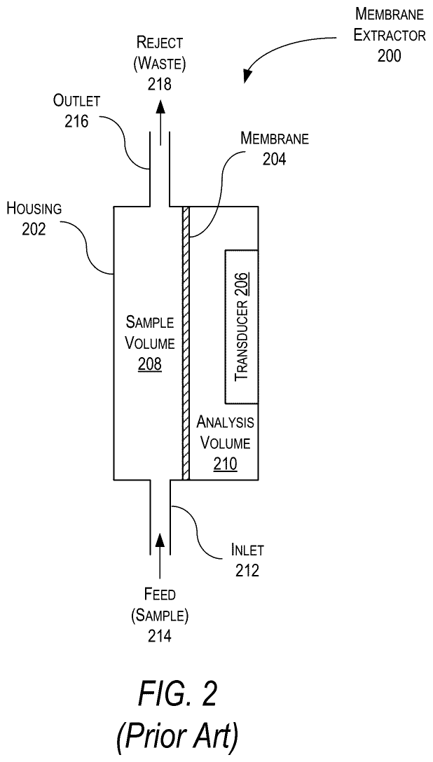

### DETAILED DESCRIPTION

Membrane inlets for chemical analysis with continuous flow sample degassing of analytes within a sample solution is disclosed. In an embodiment, the membrane inlet comprises: a housing having a sample volume and an analysis volume; a long membrane within the housing that physically separates the sample volume from the analysis volume; a sensor configured to measure a concentration for each of the analytes in the analysis volume; and a controller in signal communication with the sensor. The housing is configured to receive a flow of the sample solution through the sample volume and the long membrane is configured to permeate the at least two analytes from the sample solution into the analysis volume. The controller includes a memory, a machine-readable medium having executable instructions, and at least one processor in signal communication with the machine-readable medium, the at least one processor configured to perform operations based on the executable instructions. The operations include: producing a constant flow of the sample solution through the sample volume and over a surface of the long membrane; permeating the at least two analytes from the sample solution in the sample volume into the analysis volume as the sample solution passes along the length of the membrane until the sample solution in the sample volume has been approximately completely degassed prior to exiting an outlet of the sample volume; evacuating the analysis volume into a measurement chamber; producing a first measurement signal, with the sensor, corresponding to a first concentration of a first analyte having permeated through the membrane into the analysis volume; producing a second measurement signal, with the sensor, corresponding to a second concentration of a second analyte through the membrane into the analysis volume; and determining a ratiometric measurement for the first analyte and the second analyte based on the first measurement signal and the second measurement signal.

Also disclosed is another membrane inlet system for chemical analysis with continuous flow sample degassing of at least two analytes within a sample solution. Two or more housings each include sample volumes and analysis volumes separated by membranes, with the sample volumes and respective analysis volumes fluidically connect in a series arrangement, whereby the arrangement can combine multiple membranes to provide a longer effective length to permit more complete degassing. In an exemplary embodiment having two interconnected inlet chambers, the membrane inlet system comprises: a first housing having a first sample volume, a first analysis volume, and a first exhaust fluidically connected to the first sample volume, wherein the first housing is configured to receive a flow of the sample solution through the first sample volume and the first exhaust; a first long membrane within the first housing that physically separates the first sample volume from the first analysis volume, wherein the first long membrane is configured to permeate the at least two analytes from the sample solution into the first analysis volume; a second housing having a second sample volume, a second analysis volume, and a second exhaust, wherein the second housing is configured to receive the flow of the sample solution through the second sample volume, wherein the second analysis volume is fluidically connected to the first analysis volume; a second long membrane within the second housing that physically separates the second sample volume from the second analysis volume, wherein the second long membrane is configured to permeate the at least two analytes from the sample solution into the second analysis volume; a sensor configured to measure a concentration for each of the analytes in or having passed through the second analysis volume; and a controller in signal communication with the sensor. The controller includes a memory, a machine-readable medium having executable instructions, and at least one processor in signal communication with the machine-readable medium, the at least one processor configured to perform operations based on the executable instructions. The operations include: producing a constant flow of the sample solution through the first sample volume and over a first surface of the first long membrane; permeating the at least two analytes from the sample solution in the first sample volume into the first analysis volume until the sample solution in the first sample volume has been partially degassed prior to exiting the first outlet of the first sample volume; evacuating the first analysis volume into the second analysis volume; injecting the constant flow of the sample solution exiting the first sample volume into and through the second sample volume and over a second surface of the second long membrane; permeating the at least two analytes from the sample solution in the second sample volume into the second analysis volume until the sample solution in the second sample volume has been approximately completely degassed prior to exiting the second outlet of the second sample volume; producing a first measurement signal, with the sensor, corresponding to a first concentration a first analyte within the second analysis volume; producing a second measurement signal, with the sensor, corresponding to a second concentration of a second analyte within the second analysis volume; and determining a ratiometric measurement for the first analyte and the second analyte based on the first measurement and the second measurement.

Further disclosed is a method for chemical analysis with continuous flow sample degassing of a plurality of analytes within a first sample solution utilizing a first membrane inlet having a first housing, a first long membrane within the first housing, a second sample solution utilizing a second membrane inlet having a second housing, a second long membrane within the second housing, and a sensor, wherein the first housing has the first sample volume and a first analysis volume physically separated by the first long membrane, and the second housing has the second sample volume and a second analysis volume physically separated by the second long membrane, wherein the first housing and second housing are fluidically connected in parallel. The method comprises: producing a first constant flow of the sample solution through the first sample volume and over a first surface of the first long membrane; producing a second constant flow of the sample solution through the second sample volume and over a second surface of the second long membrane; permeating a first sub-plurality of analytes from the sample solution in the first sample volume into the first analysis volume until the sample solution in the first sample volume has been approximately completely degassed prior to exiting an first outlet of the first sample volume; permeating the second sub-plurality of analytes from the sample solution in the second sample volume into the second analysis volume until the sample solution in the second sample volume has been approximately completely degassed prior to exiting an second outlet of the second sample volume; evacuating the first analysis volume and second analysis volume; producing a first measurement signal, with the sensor, corresponding to a first concentration a first analyte through the first long membrane and second long membrane into the first analysis volume and second analysis volume; producing a second measurement signal, with the sensor, corresponding to a second concentration of a second analyte through the first long membrane and second long membrane into the first analysis volume and second analysis volume; and determining a ratiometric measurement for the first analyte and the second analyte based on the first measurement signal and the second measurement signal.

### Fixed Volume Sample Degassing

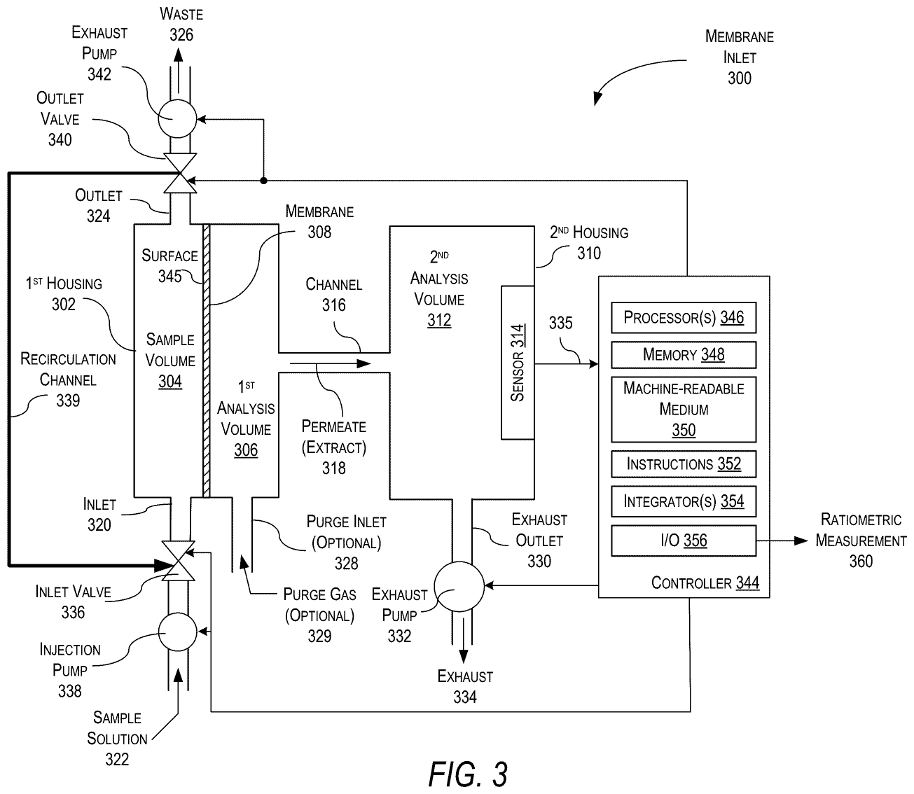

In FIG. 3, a system block diagram of an example of an implementation of membrane inlet 300 configured for fixed volume sample degassing is shown. The embodiment of FIG. 3 is an alternative solution conceived by the present Applicant and the subject of a co-pending patent application, with the embodiment disclosed herein for additional context and potential relevant teachings. In this example, the membrane inlet 300 includes a first housing 302 with a sample volume 304 and first analysis volume 306 separated by a membrane 308, and a second housing 310 having a second analysis volume 312 and a sensor 314. In this example, the first housing 302 and second housing 310 together form the housing of the membrane inlet 300. The first analysis volume 306 and the second analysis volume 312 form a combined “analysis volume” and may be physically connected by a channel 316 for the permeates (i.e., extracts of the analytes) 318 to travel from the first analysis volume 306 to the second analysis volume 312. In sample volume 304 and combined analysis volume are each fixed volume.

In this example, the first housing 302 also includes an inlet 320 to inject a sample solution 322 into the sample volume 304, an outlet 324 to eject the rejected waste 326 from the sample solution 322, and an optional purge inlet 328 for injecting an optional purge gas 329. The second housing 310 also includes an exhaust outlet 330 configured to evacuate the permeates 318 from the second analysis volume 312. As an example, the exhaust outlet 330 may be fluidically connected to an exhaust pump 332 the evacuates the permeates 318 in the second analysis volume 312 to produce exhaust 334. In this example, the sample volume 304 has a fixed and known volume.

The inlet 320 of the first housing 302 may be fluidically connected to an inlet valve 336, an injection pump 338, or both and the outlet 324 of the first housing 302 may be fluidically connected to an outlet valve 340, an exhaust pump 342, or both. In this example, the inlet valve 336, an injection pump 338, outlet valve 340, and the exhaust pump 342 are optional components that may be utilized and configured, either individually or in combination, to allow and stop the flow of the sample solution 322 through the sample volume 304 along a surface of the membrane 308. In this example, the inlet valve 336 and outlet valve 340 may be three-way valves that are in fluidically connected via a fluid channel 339.

The membrane inlet 300 also includes a controller 344. The controller 344 includes at least one processor 346, a memory 348, a machine-readable medium 350, executable instructions 352, one or more integrators 354, and an input/output module 356. The controller 344 is in signal communication with the sensor 314, exhaust pump 332, inlet valve 336, injection pump 338, outlet valve 340, and exhaust pump 342, respectively. The controller 344 may also be in signal communication with an optional injection pump (not shown) in fluidic connection with the optional purge inlet 328.

In an example of operation, when a known constant (i.e., fixed) flow of the sample solution 322 is injected into the sample volume 304, via the inlet 320, flowed over the surface 345 of the membrane 308, and evacuated, via the outlet 324, the analytes in the sample solution 322 permeate through the membrane into the analysis volume (i.e., the combination of the first analysis volume 306 and second analysis volume 312). The extracted permeate analytes 318 from the membrane 308 are then measured by the sensor 314 and constantly purged (i.e., evacuated) from the analysis volume (i.e., the combination of the first analysis volume 306 and second analysis volume 312) as an exhaust 334 with the exhaust pump 332 via the exhaust outlet 330. The resulting measurement signals 335 produced by the sensor 314 may be current signals corresponding to the amount of analytes detected by the sensor 314 that have an intensity value that is a function of time, where the sensor 314 produces a different measurement signal 335 for each analyte detected.

In this example, the controller 344 may control the injection pump 338 (if present) and/or exhaust pump 342 (if present) to control the constant flow the sample solution 322 through the sample volume 304. The controller 344 may also control the flow rate of the exhaust pump 332 that evacuates the extracted permeate 318 from the analysis volume.

Each measurement signal 335 represents the permeation flux of a detected analyte across the membrane 308 that is within the analysis volume (i.e., the combination of the first analysis volume 306 and the second analysis volume 312). It is appreciated by those of ordinary skill that that permeation flux will vary for each type of analyte because the permeation flux of an analyte through the membrane 308 is based on the properties of the membrane 308 and the diffusion characteristics of the analyte (e.g., a gas). As such, the concentration of the analyte in the analysis volume is proportional to the permeation flux of the analyte across the membrane 308.

Generally, the permeation flux through the membrane 308 may vary based on numerous complexities and the variability in permeability of membrane 308 generally causes imprecise measurements. Specifically, the rate of analyte pervaporation through the membrane 308 has dependencies on: the membrane 308 characteristics that may include, for example, material and geometry of the membrane 308; the membrane 308 boundary conditions that may develop at a surface of either side of the membrane 308; the physical conditions of the membrane 308, for example, the temperature and pressure experienced by the membrane 308; and the chemical and physical characteristics of an individual analyte species, for example, the analyte diffusion coefficients in the sample solution 322 and the membrane 308, sorption, and desorption.

However, the membrane inlet 300 is configured to analyze the concentration of the analytes in the analysis volume independent of membrane 308 permeability allowing for high precision inter-analyte ratiometric determinations and ratiometric analysis calibration techniques that are independent of the membrane 308.

In this example, the sensor 314 detects the presence of analytes in the analysis volume and produces a different measurement signal 335 for each analyte detected that represents the permeation flux of a given detected analyte across the membrane 308 that is within the analysis volume. It is appreciated by those of ordinary skill in the art that each analyte will produce a different type of measurement signal 335 because permeation flux through the membrane 308 will be different for each analyte.

In this example, the integration of each measured signal 335 may be performed by one or more integrators 354 that may be hardware circuits (such as, for example, an operational amplifier integrator circuit, application specific integrated circuit (ASIC), or other similar device) or software module that is run by the one or more processors 346 of the controller 344.

Once, the controller 344 receives the measurement signals 335, the controller 344 stops the flow of the sample solution 322 through the sample volume 304 of the housing 302. In this example, the controller 344 may stop the flow of the sample solution 322 by optionally shutting off the injection pump 338 (if present), the exhaust pump 342 (if present), and/or closing either the inlet valve 336 (if present) or outlet valve 340 (if present). Once the flow of the sample solution 322 has been stopped, all the analytes in the sample solution 322 that are trapped in the sample volume 304, given enough time, will permeate through the membrane 308 into the analysis volume, be measured by the sensor 314, and then be pumped away by the exhaust pump 322 as the exhaust 334. The controller 344 then receives the measurement signals 335 for the sensor 314 and integrates the measurement signals 335 to produce a concentration value that is proportional to the concentration of the analytes in the sample solution 322.

In this example, the controller 344 may be configured to control a rate of evacuation of the extracted permeate (i.e., the permeated plurality of analytes) from the analysis volume (i.e., the combined first analysis volume 306 and second analysis volume 312) with the exhaust pump 332. The controller 344 may also be configured to control the rate of the flow of the sample solution 322 through the sample volume 304 with the injection pump 338, outlet pump 342, or both. Furthermore, if the inlet value 336, outlet value 340, or both are configured as shut-off valves, the inlet valve 336 and/or outlet valve 340 may be configured to stop the flow of the sample solution 322 through the sample volume 304, where the controller 344 is in signal communication with the inlet valve 336 and/or outlet valve 340 and is configured to shut-off the inlet valve 336 and/or outlet valve 340.

The controller 344 may produce a ratiometric measurement value 360 that corresponds to ratio of concentration of a first analyte versus a second analyte present in the sample solution 322. The controller 344 may repeat this process for all of the analytes of interest in the sample solution 322. In this example, the ratiometric measurement value 360 may be transmitted by the input/output module 356 to an external display device (not shown).

In these examples, while the individual permeation flux through the membrane 308 for each analyte is different and dependent on many factors, the integration of these permeation fluxes produces concentration values that are independent of the membrane 308 characteristics. The ratios of these determined concentration values for each analyte are equal to the ratio of the concentrations of those different analytes within the sample solution 322. As such, by determining the permeation fluxes of the two analytes, calculating the corresponding concentration values by integrating the permeation fluxes, and dividing the calculated concentration values for each analyte, a ratiometric measurement of the analytes of the sample solution 322 can be performed accurately and relatively quickly irrespective of the membrane 308 characteristics.

In general, the membrane inlet 300 disclosed may perform three distinct methods in determining a ratiometric measurement for at least two analytes within the sample solution 322. The first method may be described as an accumulation method; the second method may be described as an integration analysis method; and the third method may be described as a recirculation method.

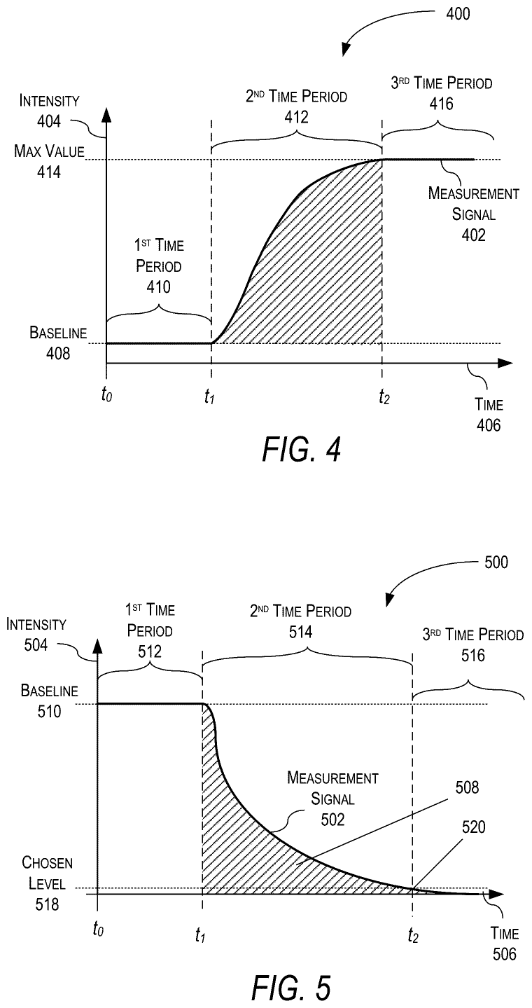

Turning to FIG. 4, a plot 400 is shown of a measured signal 402 corresponding to the measured permeation flux through the membrane 308 in an accumulation method. The plot 400 includes a vertical axis 404 representing the intensity of the measured signal 402 and a horizontal axis 406 representing time. As an example, the time values may be in minutes. In this example, the plot 400 represents an accumulation of the concentration of a given analyte within the analysis volume. If the analysis volume (i.e., the combined first analysis volume 306 and second analysis volume 312) is evacuated or purged, then sealed except for exposure to the membrane 308 interface (i.e., the surface 345), the analysis volume will fill with the analyte being extracted by the membrane 308 from the sample solution 322. Once the sample solution 322 in the sample volume 304 is completely degassed, but before an equilibrium is achieved, a measurement can then be made that is an integration of the permeated analyte.

Specifically, in this example, the measured signal 402 is shown to have a first intensity value 408 that is normalized to a baseline value for a first time period 410 that includes time t₀ to time t₁. The first time period 410 represents the period of time where the analysis volume is being purged or evacuated with the exhaust pump 332. While the analysis volume is being purged, the sensor 314 will produce the measured signal 402 with a relatively constant intensity valve that may be normalized to the baseline value 408. Once the controller 344 stops the exhaust pump 332, the analysis volume is sealed (i.e., the exhaust outlet 330 is sealed by turning off the exhaust pump 332 and no gases will be allowed to escape from the second analysis volume 312) and will no longer be purged. In a second time period 412, from time t₁ to time t₂, the analysis volume then is fixed in volume and beings to fill with an analyte of interest from the membrane 308. In the second time period 412, the sensor 314 begins to detect more molecule concentration of the analyte of interest and, resulting produces increasing intensity values for the measured signal 402 until, at time t₂, the measured signal 402 reaches a maximum value 414 representing a steady state for the measured signal 402 since sample solution 322 is completely (or approximately completely) degassed. To the extent a sample is approximately completely degassed, the target level of degassing (e.g. 99%, 98%, 95%, 90%, 85%, 80%, 75%, 70% or other levels) may be specified based on, inter alia, desired measurement accuracy and design constraints for a particular application. In this example, the controller 344 can determine that the measurement signal 402 has reached its maximum value 414 (for example, via a threshold detector). Once the maximum value 414 is reached, sample solution 322 in the sample volume 304 has been approximately completely degassed. The third time period 416, from t₂ onward, represents a steady state condition for the measured signal 402 that is proportional to the concentration of analytes of interest in the sample solution 322 and is independent of membrane 308 permeability.

Turning to FIG. 5, a plot 500 is shown of a measured signal 502 corresponding to the measured permeation flux through the membrane 308 in an integration analysis method. The plot 500 includes a vertical axis 504 representing the intensity of the measured signal 502 and a horizontal axis 506 representing time. In this example, the plot 500 represents an integration of the permeation flux of a given analyte within the analysis volume and the measured signal 502 represents the permeation flux of the given analyte within the analysis volume, where the area 508 under the measured signal 502 represents the integration of the measured signal 502.

Specifically, in this example, the measured signal 502 is shown to have a first intensity value 510 that is normalized to a baseline value for a first time period 512 that includes time t₀ to time t₁. The first time period 512 represents the period of time where the analysis volume is being purged or evacuated with the exhaust pump 332 and the sample solution 322 is flowing through the sample volume 304. In the first time period 512, the measured signal 502 produced by the sensor 314 is approximately constant at a steady state value representative of the permeation flux of the given analyte within the analysis volume. This steady state value may be normalized to a baseline value shown as the first intensity value 510. When the controller 344 shuts off the injection pump 338, outlet pump 342, inlet valve 336, or outlet valve 340, the flow of the sample solution 322 through the sample volume 304 stops, where the volume of the sample volume 304 becomes fixed (i.e., the volume amount of the sample solution 322 within the sample volume 304 becomes fixed to volume size of the sample volume 304 since it no longer is moving). The exhaust pump 332 is still purging the analysis volume of analytes so the sample solution 322 is degassed at a faster rate. In the second time period 514, from time t₁ to a time t₂, the sample solution 322 is degassed quickly until at time t₂, the sample solution 322 is approximately completely degassed. The third time period 516, form time t₂ onward, represents a complete (approximately) degassed sample solution 322.

In this example, the area 508 under the measured signal 502, at the second time period 514, represents the integration of the measured signal 502 (i.e., the permeation flux of the given analyte) that is proportional to the concentration of the analyte in the sample solution 322 and is independent of the permeability of the membrane 308. In this example, decay of the measured signal 502 is asymptotic towards a zero value of intensity which represents a steady state for the measured signal 502 since sample solution 322 is completely (or approximately completely) degassed. Since the intensity of the measured signal 502 is dropping towards zero, the method allows for an arbitrary intensity level 518 to be chosen by the controller 344 as the intensity level that represents that the sample solution 322 has almost been completely degassed. This corresponds to point 520 on the measured signal 502 at time t₂. This chosen level 518 and point 520 may be preset, or dynamically set, by the controller 344 to set the precision of the integrated concentration value as represented by the area 508 under the measured signal 502 curve. In this example, the controller 344 can determine that the measurement signal 502 has reached its chose value 518 (for example, via a threshold detector). Once the chosen value 518 is reached, the sample solution 322 in the sample volume 304 has been approximately completely degassed.

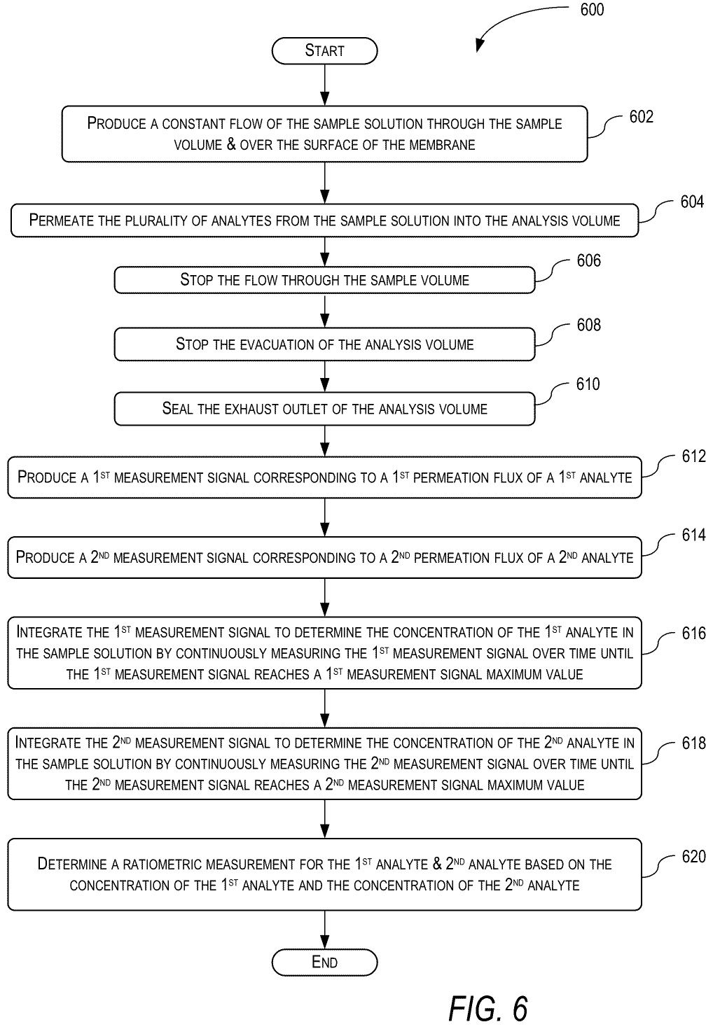

In FIG. 6, a flowchart of an example implementation of an accumulation method 600 is shown. The accumulation method 600 is performed by the membrane inlet 300 in accordance with the present disclosure. The method 600 starts by producing 602 a constant flow of the sample solution 322 through the sample volume 304 and over a surface 345 of the membrane 308 and permeating 604 the plurality of analytes from the sample solution 322 in the sample volume 304 into the analysis volume (i.e., the combined first analysis volume 306 and second analysis volume 312). The method 600 then stops 606 the flow of the sample solution 322 through the sample volume 304, stops 608 the evacuation of the analysis volume, seals 610 the exhaust outlet of the analysis volume, produces 612 a first measurement signal, with the sensor 314, corresponding to a first permeation flux of a first analyte through the membrane 308 into the analysis volume, and produces 614 a second measurement signal, with the sensor 314, corresponding to a second permeation flux of a second analyte through the membrane into the analysis volume. The method 600 then integrates 616 the first measurement signal to determine a concentration of the first analyte in the sample volume 304 by continuously measuring the first measurement signal over time until the first measurement signal reaches a first measurement signal maximum value, where the first measurement signal maximum value is proportional to the concentration of the first analyte; and integrates 618 the second measurement signal to determine a concentration of the second analyte in the sample volume 304 by continuously measuring the second measurement signal over time until the second measurement signal reaches a second measurement signal maximum value, wherein the second measurement signal maximum value is proportional to the concentration of the first analyte. The method 600 then determines 620 a ratiometric measurement for the first analyte and the second analyte in the sample solution 322 based on the concentration of first analyte and the concentration of the second analyte and then ends.

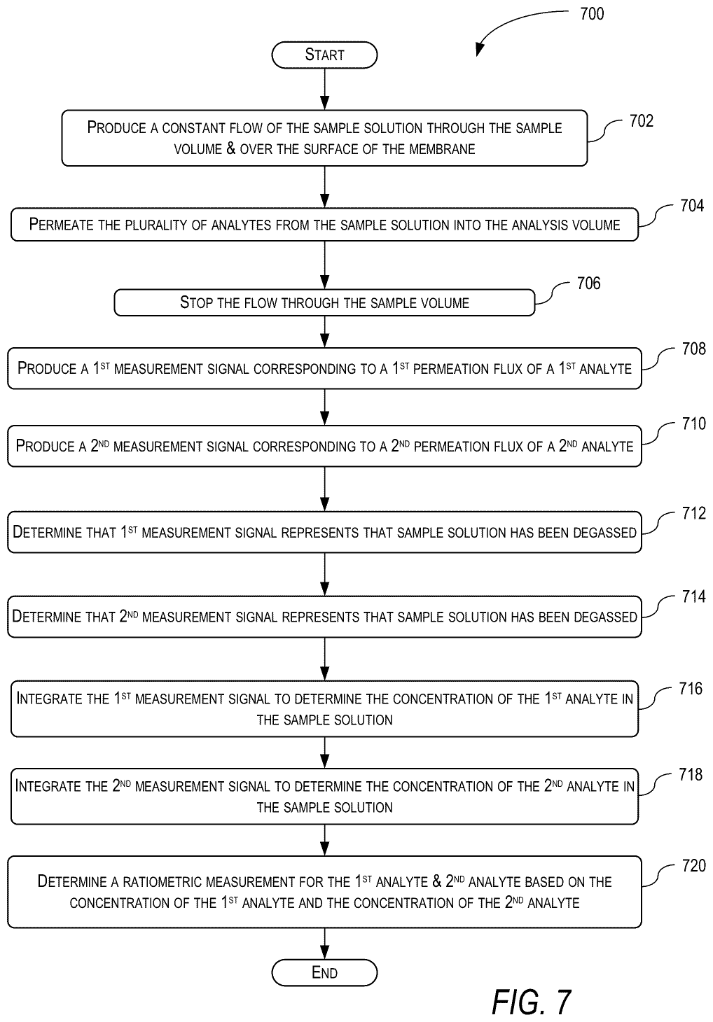

Turning to FIG. 7, a flowchart of an example implementation of an integration analysis method 700 is shown. The integration analysis method 700 is performed by the membrane inlet 300 in accordance with the present disclosure. The method 700 starts by producing 702 a constant flow of the sample solution 322 through the sample volume 304 and over a surface 345 of the membrane 308 and permeating 704 the plurality of analytes from the sample solution 322 in the sample volume 304 into the analysis volume. The method 700 then stops 706 the flow of the sample solution 322 through the sample volume 304, produces 708 a first measurement signal, with the sensor 314, corresponding to a first permeation flux of a first analyte through the membrane 308 into the analysis volume, and produces 710 a second measurement signal, with the sensor 314, corresponding to a second permeation flux of a second analyte through the membrane 308 into the analysis volume. The method then determines 712 that the first measurement signal represents that the sample solution 322 in the sample volume 304 has been approximately completely degassed; and determines 714 that the second measurement signal represents that the sample solution in the sample volume has been approximately completely degassed. The method 700 then integrates 716 the first measurement signal to determine a concentration of the first analyte in the sample volume 304, integrates 718 the second measurement signal to determine a concentration of the second analyte in the sample volume, and determines 720 a ratiometric measurement for the first analyte and the second analyte based on the concentration of first analyte and the concentration of the second analyte. The method 700 then ends.

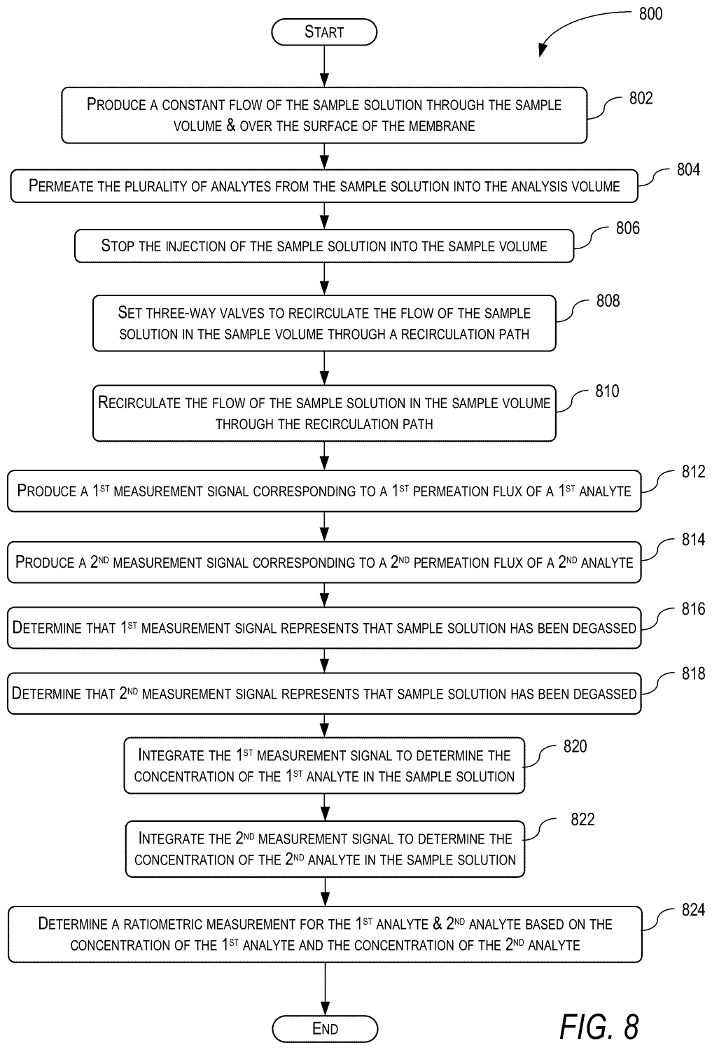

In FIG. 8, a flowchart of an example implementation of a recirculation method 800 is shown. The recirculation method 800 is performed by the membrane inlet 300 in accordance with the present disclosure. The method 800 starts by producing 802 a constant flow of the sample solution 322 through the sample volume 304 and over a surface 345 of the membrane 308 and permeating 804 the plurality of analytes from the sample solution 322 in the sample volume 304 into the analysis volume. The method 800 then stops the flow of the sample solution 322 through the sample volume 304 by stopping 806 the injection of the sample solution 322 into the sample volume 304 and switching (i.e., setting) 808 the first three-way valve and second three-way valve to recirculate the flow of the sample solution 322 in the sample volume 304 through the recirculation path. The method 800 then recirculates 810 the flow of the sample solution 322 in the sample volume 304 through a recirculation path that includes the sample volume 304, the first three-way valve 336, recirculation channel 339, and the second three-way valve 340. The method 800 then produces 812 a first measurement signal, with the sensor 314, corresponding to a first permeation flux of a first analyte through the membrane 308 into the analysis volume; produces 814 a second measurement signal, with the sensor 314, corresponding to a second permeation flux of a second analyte through the membrane 308 into the analysis volume; determines 816 that the first measurement signal represents that the sample solution in the sample volume has been approximately completely degassed; determines 818 that the second measurement signal represents that the sample solution in the sample volume has been approximately completely degassed; integrates 820 the first measurement signal to determine a concentration of the first analyte in the sample volume 304; integrates 822 the second measurement signal to determine a concentration of the second analyte in the sample volume 304; and determines 824 a ratiometric measurement for the first analyte and the second analyte based on the concentration of first analyte and the concentration of the second analyte. The method 800 then ends.

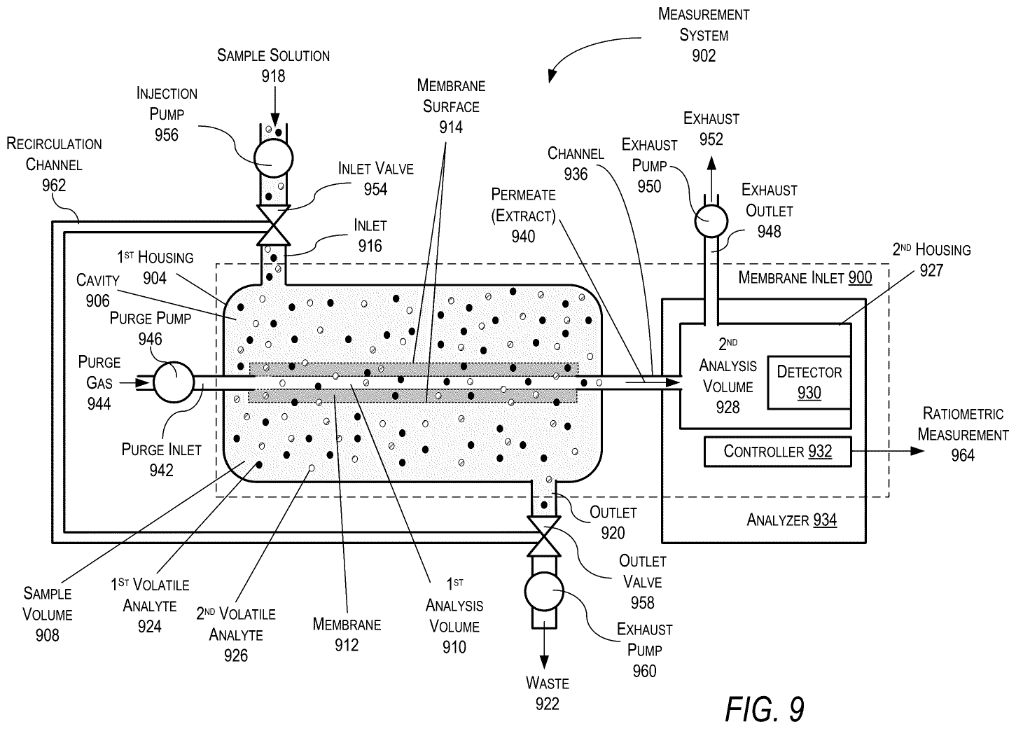

FIG. 9 is a system block diagram of an example implementation of another membrane inlet 900 within a measurement system 902 in accordance with the present disclosure. In this example, the membrane inlet 900 includes a first housing 904 with a cavity 906. The cavity 906 includes a sample volume 908, a first analysis volume 910, and a membrane 912 separating the first analysis volume 910 from the sample volume 908. In this example, the first analysis volume 910 is a membrane 912 capillary and the membrane 912 includes a membrane surface 914 that is exposed to the sample volume 908 in the cavity 906. In this example, the membrane 912 is cylindrical in shape having a membrane 912 diameter and the first housing 904 is also cylindrical having a larger diameter than the membrane 912 diameter. The first housing 904 has an inlet 916 for receiving a sample solution 918 into the sample volume 908 and an outlet 920 for removing waste 922 of the sample solution 918 from the sample volume 908. In this example, the sample solution 918 may include a plurality of volatile analytes but in order to simplify the illustration, only two analytes are shown that include first volatile analyte 924 and second volatile analyte 926.

The membrane inlet 900 also includes a second housing 927 having a second analysis volume 928, a detector (i.e., sensor) 930, and a controller 932. In this example, the second analysis volume 928, detector 930, and controller 932 may be part of an analyzer 934. In this example, the second analysis volume 928 is fluidically connected to the first analysis volume 910 via a channel 936 where the first analysis volume 910 and the second analysis volume 928 form a combined “analysis volume” and may be physically connected by the channel 936 for the permeates (i.e., extracts of the analytes) 940 to travel from the first analysis volume 910 to the second analysis volume 928. In this example, the sample volume 908 and combined analysis volume are each fixed volumes.

The analyzer 934 may be, for example, a spectroscopy analyzer or mass spectrometer that utilizes mass spectrometry (MS) and may include a vacuum chamber (not shown) having an electron source (not shown), an accelerator section (not shown), deflection electromagnets (not shown), outlet (not shown) to a vacuum pump, and the detector 930. The analyzer 934 may also include the controller 932.

In this example, the first housing 904 may also include an optional purge inlet 942 for injecting an optional purge gas 944 via a purge pump 946. The second housing 927 also includes an exhaust outlet 948 configured to evacuate the permeates 940 from the second analysis volume 928. As an example, the exhaust outlet 948 may be fluidically connected to an exhaust pump 950 the evacuates the permeates 940 in the second analysis volume 928 to produce exhaust 952. In this example, the sample volume 908 has a fixed volume.

The inlet 916 of the first housing 904 may be fluidically connected to an inlet valve 954, an injection pump 956, or both and the outlet 920 of the first housing 904 may be fluidically connected to an outlet valve 958, an exhaust pump 960, or both. In this example, the inlet valve 954, injection pump 956, outlet valve 958, and the exhaust pump 960 are optional components that may be utilized and configured, either individually or in combination, to allow and stop the flow of the sample solution 918 through the sample volume 908 along the surface 914 of the membrane 912. In this example, the inlet valve 954 and outlet valve 958 may be three-way valves that are in fluidically connected via a fluid channel 962.

While not shown for the purpose of simplicity of illustration, it is appreciated by those of ordinary skill in the art that controller 932 is in signal connection with measurement system 902, exhaust pump 950, purge pump 946, inlet valve 954, injection pump 956, outlet valve 958, and exhaust pump 960 as was similarly described in relation to FIG. 3. Similar to the controller 344 described in FIG. 3, controller 932 also includes at least one processor, a memory, a machine-readable medium, executable instructions, one or more integrators, and an input/output module. Furthermore, the controller 932 may produce a ratiometric measurement value 964 that corresponds to ratio of concentration of a first analyte (i.e., first volatile analyte 924) versus a second analyte (i.e., second volatile analyte 926) present in the sample solution 918. The controller 932 may repeat this process for all of the analytes of interest in the sample solution 918. In this example, the ratiometric measurement value 964 may be transmitted by the input/output module (not shown) to an external display device (not shown).

Similar to the membrane inlet 300, the membrane inlet 900 may operate performing the three distinct methods in determining the ratiometric measurement 964 for the two volatile analytes 924 and 926 within the sample solution 918. Again, the first method may be the accumulation method (described in FIGS. 4 and 6); the second method may be the integration analysis method (described in FIGS. 5 and 7); and the third method may be the recirculation method that is described in FIG. 8.

An additional method that may be performed by either the membrane inlet 300 or membrane inlet 900 includes injecting the purge gas (329 or 944) into the analysis volume via the purge inlet (328 or 942) and evacuating the analysis volume via the exhaust outlet (330 or 948), where evacuating the analysis volume includes evacuating the permeated plurality of analytes (318 or 940) from the sample solution (322 or 918) and the purge gas (329 or 944).

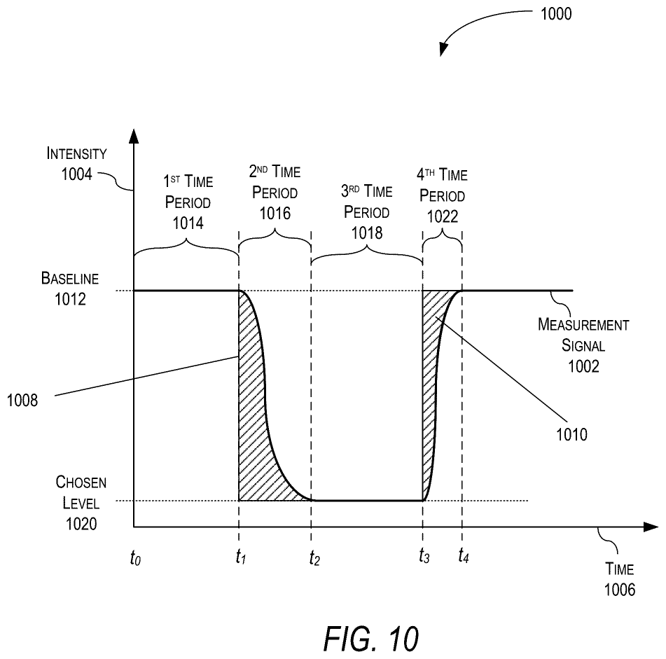

Turning to FIG. 10, a plot 1000 of a measured signal 1002 corresponding to the measured permeation flux through the membrane 308 is shown in another integration method in accordance with the present disclosure. The plot 1000 includes a vertical axis 1004 representing the intensity of the measured signal 1002 and a horizontal axis 1006 representing time. In this example, the plot 1000 represents a difference in integrations of the permeation flux of a given analyte within the analysis volume and the measured signal 1002 represents the permeation flux of the given analyte within the analysis volume, where the areas below (1008) and above (1010) the measured signal 1002 represents the integration of the measured signal 1002.

As described earlier, in this example, the measured signal 1002 is shown to have a first intensity value 1012 that is normalized to a baseline value for a first time period 1014 that includes time t₀ to time t₁. The first time period 1014 represents the period of time where the analysis volume is being purged or evacuated with the exhaust pump 332 and the sample solution 322 is flowing through the sample volume 304. In the first time period 1014, the measured signal 1002 produced by the sensor 314 is approximately constant at a steady state value representative of the permeation flux of the given analyte within the analysis volume. This steady state value may be normalized to the baseline value shown as the first intensity value 1012. When the controller 344 shuts off the injection pump 338, outlet pump 342, inlet valve 336, or outlet valve 340, the flow of the sample solution 322 through the sample volume 304 stops, where the volume of the sample volume 304 becomes fixed (i.e., the volume amount of the sample solution 322 within the sample volume 304 becomes fixed to volume size of the sample volume 304 since it no longer is moving). The exhaust pump 332 is still purging the analysis volume of analytes so the sample solution 322 is degassed at a faster rate. In the second time period 1016, from time t₁ to a time t₂, the sample solution 322 is degassed quickly until at time t₂, the sample solution 322 is approximately completely degassed. The third time period 1018, form time t₂ to t₃, represents a complete (approximately) degassed sample solution 322 where the measured signal 1002 intensity drops to a chosen level 1020. However, in this example, when the pump (either injection pump 338 or exhaust pump 342) of the sample volume 304 is turned off (similar to the examples described earlier), if the volume of the membrane 308 or 912 (i.e., the physical volume defined by the size of the membrane 308) has a volume that is of the same order of magnitude as the sample volume 304 or 908, there may need to be a correction to account for permeate contained in the membrane 308 or 912. In this example, referring to FIG. 9, the membrane 912 may have a radius that defines a combination volume that equals the volume of the membrane 912 and the first analysis volume 910, where an inner radius of the membrane defines the first analysis volume 910 and the radius of membrane 912 minus the inner radius defines the cylindrical volume of the membrane 912 that surrounds the first analysis volume 910.

In this case, because the content of permeate in the membrane 308 is non-negligible, the area 1008 under the curve (i.e., measurement signal 1002) from the moment the volume (i.e., the sample volume 304) is fixed, the amount of analyte in the analysis volume is proportional to all the permeate in the membrane 308 plus all the permeate in the sample volume 304. In this example, the exhaust pump 332 is continuously evacuating the analysis volume. When the pump (i.e., either injection pump 338 or exhaust pump 342) is then turned back on, a similar measurement is made with the upward rising data (i.e., the measurement signal 1002) at a fourth time period 1022 from time t₃ to t₄. The negative space (i.e., area 1010 under the curve) from the integration of the pump-on data (i.e., the measurement signal 1002 at the fourth time period 1022) is proportional to the amount of analytes that was sorbed by the membrane 308. The difference between the first integration value (i.e., the first area 1008) and second integration value (i.e., the second area 1010 (provides a correction to determine the precise amount of analyte that was in the sample solution 322 only that was in the sample volume 304.

A benefit of the disclosed membrane inlets (300 or 900) is that they do not have to be calibrated in situ with special calibration solutions injected as a sample solution into the membrane inlets (300 or 900) because the any calibration is independent of the membrane (308 or 912) conditions. As an example, the purge gas (329 or 944) may be utilized as calibration gas because it is injected behind the membrane (308 or 912) meaning that the calibration could be done with a gas reference that is only injected into the analysis volume of the membrane inlets (300 or 900).

### Continuous Flow Volume Sample Degassing

While the discontinuous, fixed-volume degassing approaches described above may be desirable in some applications, in other circumstances it may be preferred to implement a continuous flow measurement system. That said, continuous flow techniques for ratiometric measurements of different analytes can be challenging to the extent that, for example, different analytes permeate through a membrane at different rates. Moreover, the permeation rates may vary based on pressure and other conditions, which may be difficult or impossible to reliably control for in certain in situ applications. Therefore, ratiometric measurements may be inaccurate in many continuous flow systems.

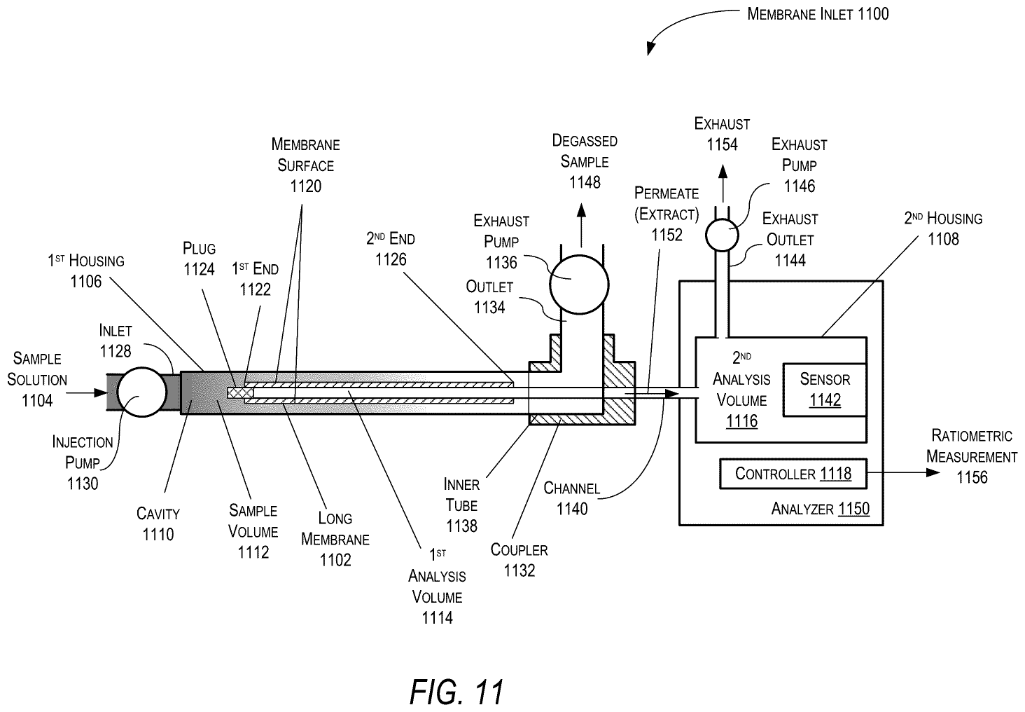

FIG. 11 is a system block diagram of an example of an implementation of another membrane inlet 1100 utilizing continuous sample flow over a long and/or very thin membrane 1102 (herein generally referred to simply as a “long membrane”) to approximately completely degas a sample solution 1104 in accordance with the present disclosure. In this example, the long membrane 1102 may be “long” or very thin membrane. The length and/or thickness of the long membrane 1102 is configured to extensively degas the sample solution 1104 before it leaves the membrane inlet 1100. In general, the long membrane 1102 is a “high extraction” membrane that is configured has a high total analyte flux relative to the supplied sample solution injected into the sample volume such that there is more analytes being permeated through the membrane than what is being supplied by the flowing sample solution through the sample volume. By using a long membrane 1102 to completely or nearly-completely degas a sample during the course of its journey through a membrane inlet, it may be possible to make reliable ratiometric measurements in a wide range of dynamic in situ applications. To the extent a sample is approximately completely degassed, the target level of degassing (e.g. 99%, 98%, 95%, 90%, 85%, 80%, 75%, 70% or other levels) may be specified based on, inter alia, desired measurement accuracy and design constraints for a particular application.

In this example, the membrane inlet 1100 includes a first housing 1106, second housing 1108, a cavity 1110 within the first housing 1106, a sample volume 1112 within the cavity 1110, a first analysis volume 1114 within the cavity 1110 and separated from the sample volume 1112 by the long membrane 1102, a second analysis volume 1116 within the second housing 1108, and a controller 1118. The long membrane 1102 includes a membrane surface 1120 that may be terminated at a first end 1122 of the long membrane 1102 by a plug 1124 or thermocouple (for measuring the temperature of the first analysis volume 1114). The long membrane 1102 also includes a second end 1126 opposite the first end 1122. The first housing 1106 also includes an inlet 1128 that may be fluidically connected to an injection pump 1130. The first housing 1106 is connected to a fluidic coupler 1132 (such as, for example a tee) having three ports. The fluidic coupler 1132 is fluidically connected to the sample volume 1112 and an outlet 1134 that may be fluidically connected to a exhaust pump 1136. The fluidic coupler 1132 may also include an inner tube 1138, within the fluidic coupler 1132, that is fluidically connected to the first analysis volume 1114 and a channel 1140, where the channel 1140 is fluidically connected to the second analysis volume 1116. The second analysis volume 1116 includes a sensor 1142 and an exhaust outlet 1144. The exhaust outlet 1144 may be fluidically connected to an exhaust pump 1146.

In this example, the membrane inlet 1100 shown is similar to the membrane inlets described in relation to FIGS. 3 and 9, except that the long membrane 1102 may be longer or thinner than the membranes 304 and 912 and is configured for high analyte total analyte fluc to significantly degas the sample solution 1104. For purposes of this embodiment, a long membrane is a membrane having sufficient analyte extraction to degas a sample to a threshold level during the course of its flow along the membrane surface. The exact membrane geometry required may vary depending on, e.g., the membrane material, thickness, length, the specific analyte compounds of interest, the sample flow rate, and ambient conditions. Length perhaps the easiest membrane parameter to optimize for various use cases, as such, commercial membranes will be characterized to permit ready calculation of required membrane length for a particular level of degassing of particular analytes. While it may be desirable to select a membrane material and length that provides for complete degassing of all analytes of interest (e.g. near 100% degassing), in some applications that may not be feasible with respect to e.g. cost or form factor. However, a threshold level of degassing may be considered complete or sufficiently complete for purposes of a desired measurement accuracy. For example, if a membrane is specified to provide 100% degassing of a first analyte and at least 90% degassing of a second analyte, the use of the 90% threshold defines a maximum level of inaccuracy for a resulting ratiometric measurement of concentration for the two analytes of interest. In various embodiments and applications, different degassing thresholds may be specified for purposes of determining long membrane geometry, such as: 95%, 90%, 85%, or 80% degassed.

In this example, the first analysis volume 1114 is a long membrane 1102 capillary and the long membrane 1102 includes the membrane surface 1120 that is exposed to the sample volume 1112 in the cavity 1110. The long membrane 1102 may be cylindrical in shape having a long membrane 1102 diameter and the first housing 1106 may also be cylindrical having a larger diameter than the long membrane 1102 diameter.

In this example, the second analysis volume 1116, sensor 1142, and controller 1118 may be part of an analyzer 1150. The second analysis volume 1116 is fluidically connected to the first analysis volume 1114 via the inner tube 1138 and channel 1140 where the first analysis volume 1114 and the second analysis volume 1116 form a combined “analysis volume” and may be physically connected by the inner tube 1138 and channel 1140 for the permeates (i.e., extracts of the analytes) 1152 to travel from the first analysis volume 1114 to the second analysis volume 1116. The analyzer 1150 may be, for example, a spectroscopy analyzer or mass spectrometer that utilizes mass spectrometry (MS) and may include a vacuum chamber (not shown) having an electron source (not shown), an accelerator section (not shown), deflection electromagnets (not shown), outlet (not shown) to a vacuum pump, and the sensor 1142. The analyzer 1150 may also include the controller 1118.

Utilizing the membrane inlet 1100, the sample solution 1104 may be optionally partially degassed or almost (i.e., approximately) completely degassed because the length of the long membrane 1102 is such that the analytes in the sample solution 1104 will diffuse almost completely into the first analysis volume 1114 as the sample solution 1104 flows through the sample volume 1112 along the membrane surface 1120 from the first end 1122 to the second end 1126 of the long membrane 1102.

In an example of operation, the shading in FIG. 11 shows how the concentration of analytes in the sample solution 1104 are highest at the inlet 1128 and first end 1122 of the sample volume 1112 and the concentration is gradually reduced as the sample solution 1104 flows from the inlet 1128 to the second end 1126, where the concentration of analytes in the sample solution 1104 at the second end 1126 is approximately zero because the sample solution 1104 has been approximately completely degassed into the first analysis volume 1114. The degassed (or approximately degassed) sample 1148 is then exhausted through the outlet 1134 via the exhaust pump 1136. The extracted permeate analytes 1152 from the long membrane 1102 are then measured by the sensor 1142 and constantly purged (i.e., evacuated) from the analysis volume (i.e., the combination of the first analysis volume 1114 and second analysis volume 1116) as an exhaust 1154 with the exhaust pump 1146 via the exhaust outlet 1144. The resulting measurement signals produced by the sensor 1142 may be current signals corresponding to the amount of analytes detected by the sensor 1142 that have an intensity value that is a function of time, where the sensor 1142 produces a different measurement signal for each analyte detected. The measurement signals for each analyte represents the corresponding concentration of the analytes such that a radiometric measurement 1156 may be determined from the different measurement signals.

In general, the membrane inlet 1100 performs a method that produces a constant flow of the sample solution 1104 through the sample volume 1112 and over the long membrane surface 1120 while the analysis volume (i.e., the combined first analysis volume 1114 and second analysis volume 1116) is evacuated by the exhaust pump 1146. The plurality of analytes in the sample solution 1104 are then permeated from the sample solution 1104 in the sample volume 1112 into the analysis volume until the sample solution 1104 in the sample volume 1112 has been approximately completely degassed prior to exiting the outlet 1132 of the sample volume 1112 via the fluidic coupler 1132. The extracted permeate 1152 is then passed from the first analysis volume 1114 to the second analysis volume 1116 via the inner tube 1138 and channel 1140. The sensor 1142 then measures the concentration of the analytes in the analysis volume and produces a first measurement signal corresponding to a first concentration a first analyte through the long membrane 1102 into the analysis volume and a second measurement signal corresponding to a second concentration of a second analyte through the long membrane 1102 into the analysis volume. The controller 1118 then determines the ratiometric measurement 1156 for the first analyte and the second analyte based on the concentration of first analyte and the concentration of the second analyte.

In this example, the length of the long membrane 1102 is determined by the desired precision of the ratiometric measurement 1156 and the type of analytes that are to be measured. Generally, an optimal length for the long membrane 1102 is determined by what type of analytes are being measured and the disparities between the type of analytes. If the analytes are chemically similar, the length of the long membrane 1102 may be determined to be a length long enough to completely degas a given analyte of interest.

As an example, if there is a desire to compare two analytes such as, for example, Methane and Butane, it is noted that Butane will typically have a generally slower diffusion rate through a given membrane when compared to Methane. For example, a long membrane 1102 that is approximately six inches long may extract approximately 90 percent of Methane, while only extracting about 60 percent of Butane. This would cause approximately a 30 percent error in the ratiometric measurement 1156. In this example, if the long membrane 1102 is made longer, the extraction of the Methane may be increased to 95 percent, while the extraction of the Butane will increase to approximately 80 percent. This new example would result in a 15 percent error instead of the original 30 percent error. In this example, as the length of the long membrane 1102 is increased, the extraction of both analytes will converge towards 100 percent and the resulting error in the ratiometric measurement 1156 will drop towards zero.

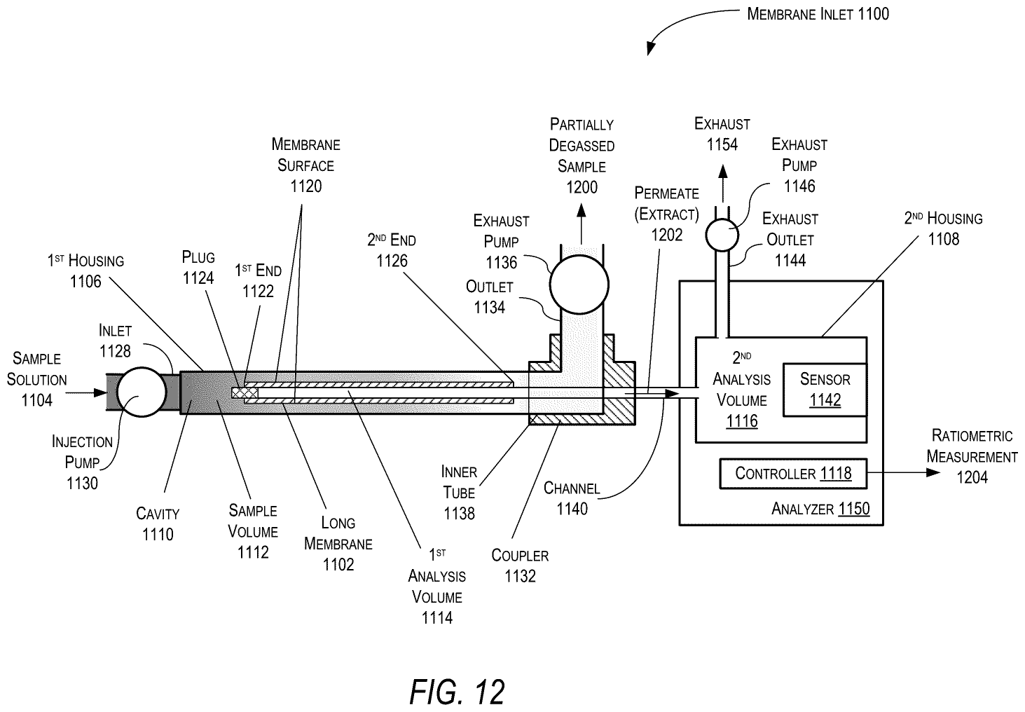

Turning to FIG. 12, the membrane inlet 1100 is shown where the sample solution 1104 is only partially degassed in accordance with the present disclosure. In an example of operation, in this example, the sample solution 1104 is injected into the inlet 1128 and the sample solution 1104 flows through the sample volume 1112 over the membrane surface 1120 but the sample solution 1104 is only partially degassed and exhausted from the outlet 1134 as a partially degassed sample 1200. Again the shading in FIG. 12 shows how the concentration of analytes in the sample solution 1104 are highest at the inlet 1128 and first end 1122 of the sample volume 1112 and the concentration is gradually reduced as the sample solution 1104 flows from the inlet 1128 to the second end 1126, where the concentration of analytes in the sample solution 1104 at the second end 1126 is only partially degassed because the sample solution 1104 has not been completely degassed into the first analysis volume 1114 because the length of the long membrane 1102 is not long enough to completely degas the analytes in the sample solution 1104. The partially degassed sample 1200 is then exhausted through the outlet 1134 via the exhaust pump 1136. The extracted permeate analytes 1202 from the long membrane 1102 are then measured by the sensor 1142 and constantly purged (i.e., evacuated) from the analysis volume (i.e., the combination of the first analysis volume 1114 and second analysis volume 1116) as an exhaust 1154 with the exhaust pump 1146 via the exhaust outlet 1144. The resulting measurement signals produced by the sensor 1142 correspond to the amount of analytes detected by the sensor 1142 and represent the corresponding concentration of the analytes such that the ratiometric measurement 1204 may be determined from the different measurement signals.

This example may be utilized to measure analytes that are chemically similar such as, for example, isotopes where complete (or approximately complete) degassing is not necessary for accurate ratiometric measurements. As an example, if the two Methane (such as, for example, ¹²CH₄ and ¹³CH₄) analytes are to be measured, these isotopes are isomers that are very similar and share similar diffusion characteristics. Generally, if the length of the long membrane 1102 is only long enough to maybe produce approximately 1 percent of extraction, the resulting ratiometric measurement 1204 will have significant bias error. However, since both analytes are very similar, if the length of the long membrane 1102 is extended so as to maybe produce approximately 30 to 40 percent extraction of the analytes, the bias error in the ratiometric measurement 1204 will decrease significantly such that ratiometric measurement 1204 will have sufficient precision for most applications. As such, this partial degassing approach will provide sufficient precision ratiometric measurements 1204 without having to fully (i.e., completely) degas the sample solution 11104.

In some applications, it may be costly, difficult, or impossible to procure a single membrane capable of achieving desired threshold levels of degassing for analytes of interest. In such circumstances, however, it may be possible to utilize a membrane inlet system that connect together multiple chambers, each with its own membrane. Combined in sequence or parallel (or combination thereof), such an embodiment may present more desirable degassing levels, and/or improved cost or form factor.

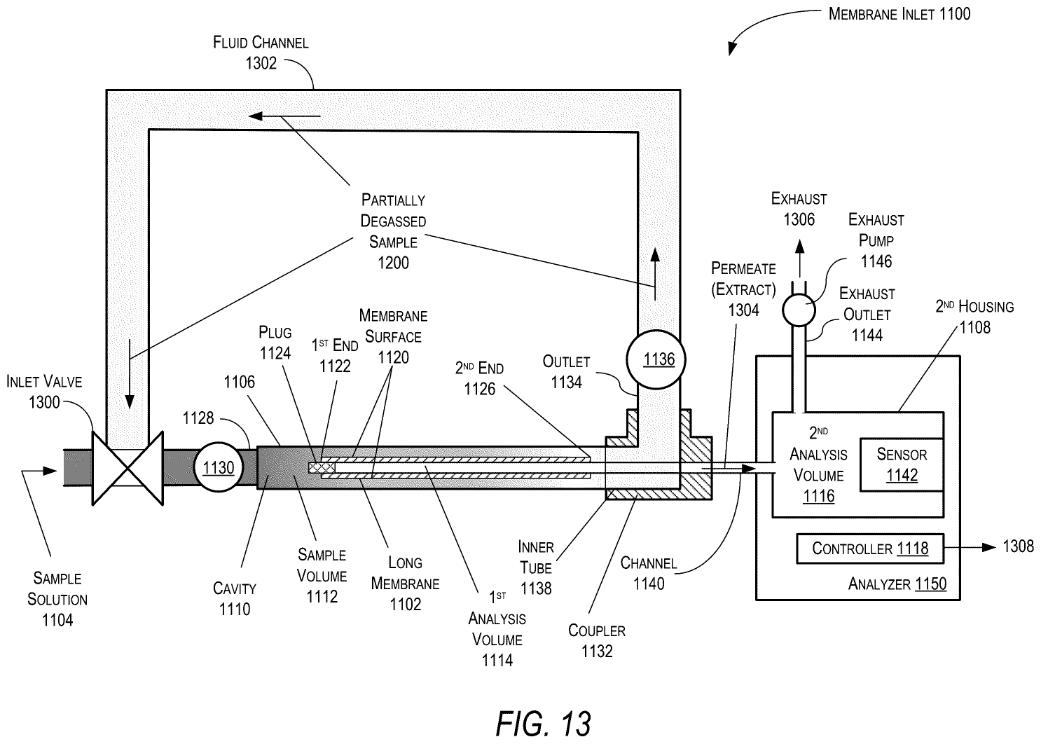

In FIG. 13, the system block diagram shown in FIG. 12 is shown where the sample solution is recirculated through the membrane inlet 1100. In this example, the inlet 1128 of the first housing 1106 may be fluidically connected to an inlet valve 1300, the injection pump 1130, or both and the outlet 1134 of the first housing 1106 may be fluidically connected to an optional outlet valve (not shown), the exhaust pump 1134, or both. In this example, the inlet valve 1300, injection pump 1130, optional outlet valve, and the exhaust pump 1136 may be optional components that may be utilized and configured, either individually or in combination, to allow the flow of the sample solution 1104 through the sample volume 1112 along the surface of the membrane 1102, through an optional fluid channel 1302 back to the inlet 1128 via a fluid channel 1302 and the inlet valve 1300. In this example, the inlet valve 1300 and optional outlet valve may be three-way valves that are fluidically connected via the fluid channel 1302.

In this example, the inlet valve 1300 may stop the flow of sample solution 1104 into the sample value 1112 and the partially degassed sample 1200 (described in relation to FIG. 12) may be recirculated through the sample volume 1112, via the fluid channel 1302 and inlet valve 1300, iteratively until the sample solution 1104 that was originally injected into the sample volume 1112 is approximately completely degassed in a similar fashion described in relation to FIGS. 3, 8, and 9 The extracted permeate analytes 1304 from the long membrane 1102 are then measured by the sensor 1142 and constantly purged (i.e., evacuated) from the analysis volume (i.e., the combination of the first analysis volume 1114 and second analysis volume 1116) as an exhaust 1306 with the exhaust pump 1146 via the exhaust outlet 1144. The resulting measurement signals produced by the sensor 1142 correspond to the amount of analytes detected by the sensor 1142 and represent the corresponding concentration of the analytes such that the ratiometric measurement 1308 may be determined from the different measurement signals.

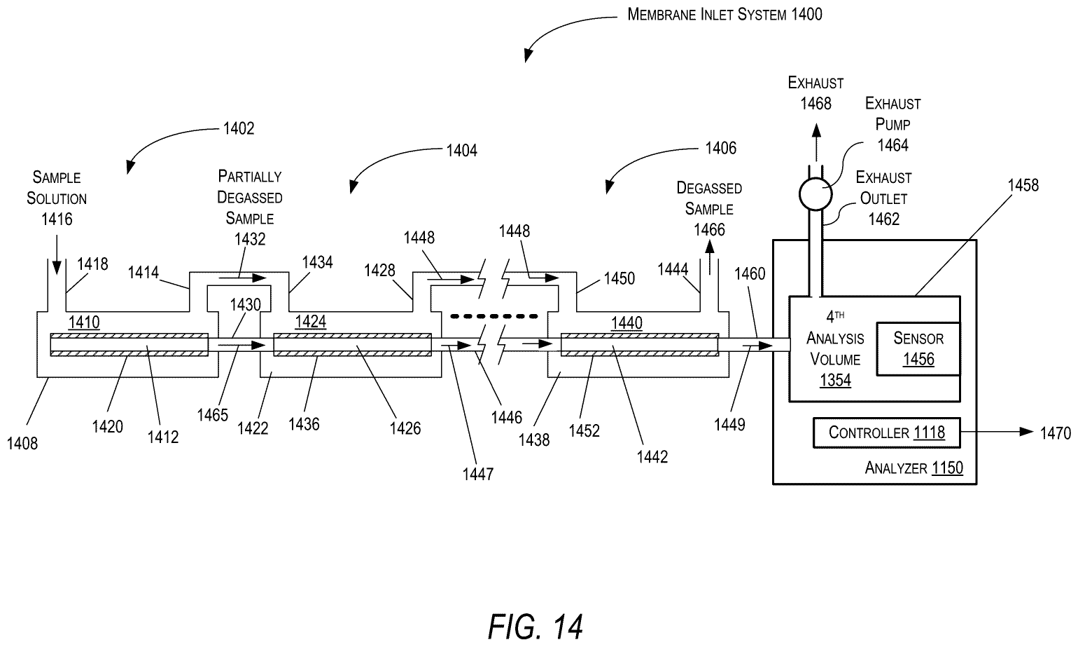

FIG. 14 is a system block diagram of an example of a sequential implementation of a membrane inlet 1400 system utilizing multiple membrane inlets 1402, 1404 and 1406 in accordance with the present disclosure. In this example, there may be any number of membrane inlets but for the ease of illustration only three membrane inlets 1402, 1404, and 1406 are shown. For purposes of ease of illustration, only sample volumes, analysis volumes, inlets, outlets, membranes, and channels from the analysis volumes are shown. Specifically, the membrane inlet system 1400 includes the first membrane inlet 1402 including a first housing 1408 having a first sample volume 1410, a first analysis volume 1412, and a first outlet 1414 fluidically connected to the first sample volume 1410, where the first housing 1408 is configured to receive a flow of the sample solution 1416 through the first sample volume 1410 and out through the first outlet 1414. The sample solution 1416 being injected into the sample volume 1410 at an inlet 1418. The first membrane inlet 1402 further including a first long membrane 1420 within the first housing 1408 that physically separates the first sample volume 1410 from the first analysis volume 1412, where the first long membrane 1420 is configured to permeate the at least two analytes from the sample solution 1416 into the first analysis volume 1412.

The second membrane inlet 1404 includes a second housing 1422 having a second sample volume 1424, a second analysis volume 1426, and a second outlet 1428, where the second housing 1422 is configured to receive the flow of the sample solution 1416 through the second sample volume 1424, wherein the second analysis volume 1426 is fluidically connected to the first analysis volume 1412 via a first channel 1430. The sample solution 1416 being exhausted as a partially degassed sample 1432 that is injected into the second sample volume 1424 via a second inlet 1434. The second membrane inlet 1404 further including a second long membrane 1436 within the second housing 1422 that physically separates the second sample volume 1424 from the second analysis volume 1426, where the second long membrane 1436 is configured to permeate the at least two analytes from the partially degassed sample 1432 into the second analysis volume 1426.

The third membrane inlet 1406 includes a third housing 1438 having a third sample volume 1440, a third analysis volume 1442, and a third outlet 1444, where the third housing 1438 is configured to receive the flow of the sample solution 1416 through the third sample volume 1440, where the third analysis volume 1442 is fluidically connected to the second analysis volume 1426 via a second channel 1446. The partially degassed sample 1432 being exhausted as a further partially degassed sample 1448 that is injected into the third sample volume 1440 via a third inlet 1450. The third membrane inlet 1406 further including a third long membrane 1452 within the third housing 1438 that physically separates the third sample volume 1440 from the third analysis volume 1442, where the third long membrane 1452 is configured to permeate the at least two analytes from the further partially degassed sample 1448 into the third analysis volume 1442.

The membrane inlet system 1400 further includes a fourth analysis volume 1454 having a sensor 1456 configured to measure the concentration for each of the analytes in the fourth analysis volume 1454. In this example, the fourth analysis volume 1454 is within a fourth housing 1458 and the third analysis volume 1442 is fluidically connected to the fourth analysis volume 454 via a third channel 1460. The fourth analysis volume 1354 has an exhaust outlet 1462 fluidically connected to an exhaust pump 1464.

In an example of operation, the controller 1118 performs a method that includes producing a constant flow of the sample solution 1416 through the first sample volume 1410 and over a first surface of the first long membrane 1420 and out the first outlet 1414 as the partially degassed sample 1432; and evacuating a first permeate 1465 from the first analysis volume 1412 into the second analysis volume 1426. The method then includes permeating the at least two analytes from the sample solution 1416 in the first sample volume 1410 into the first analysis volume 1412 until the sample solution 1416 in the first sample volume 1410 has been partially degassed prior to exiting the first outlet 1414 of the first sample volume 1410 producing the partially degassed sample 1432. The partially degassed sample 1432 is then injected into the inlet 1434 as a constant flow of the sample solution 1416 (but partially degassed) and through the second sample volume 1424 and over a second surface of the second long membrane 1436. The at least two analytes from the partially degassed sample 1432 in the second sample volume 1424 are then permeated into the second analysis volume 1426 until the partially degassed sample 1432 in the second sample volume 1424 has been further degassed prior to exiting the second outlet 1428 of the second sample volume 1424 to produce the further partially degassed sample 1448. A combined permeate 1467 that includes the first permeate 1465 and a second permeate from the second long membrane 1436 is evacuated into the third analysis volume 1442. The further partially degassed sample 1448 is then injected into the third sample volume 1440 via the third inlet 1450. The further partially degassed sample 1448 is then flowed through the third sample volume 1440 and over a third surface of the third long membrane 1452. The at least two analytes from the further partially degassed sample 1448 in the third sample volume 1440 are then permeated into the third analysis volume 1442 until the further partially degassed sample 1448 in the third sample volume 1440 has been approximately completely degassed prior to exiting the third outlet 1444 of the third sample volume 1440 as the degassed sample 1466.

In this example, the total permeate analytes 1449 are excavated from the first analysis volume 1412 through the third analysis volume 1442 into the fourth analysis volume 1454 and out the exhaust outlet 1462 as exhaust 1468 by the exhaust pump 1464. The sensor 1456 then measures a first concentration of the first analyte and the second concentration of the second analyte through the combined first long membrane 1420, second long membrane 1436, and third long membrane 1452 into the fourth analysis volume 1454. The controller 1118 then determines a ratiometric measurement 1470 for the first analyte and the second analyte based on the concentration of first analyte and the concentration of the second analyte.

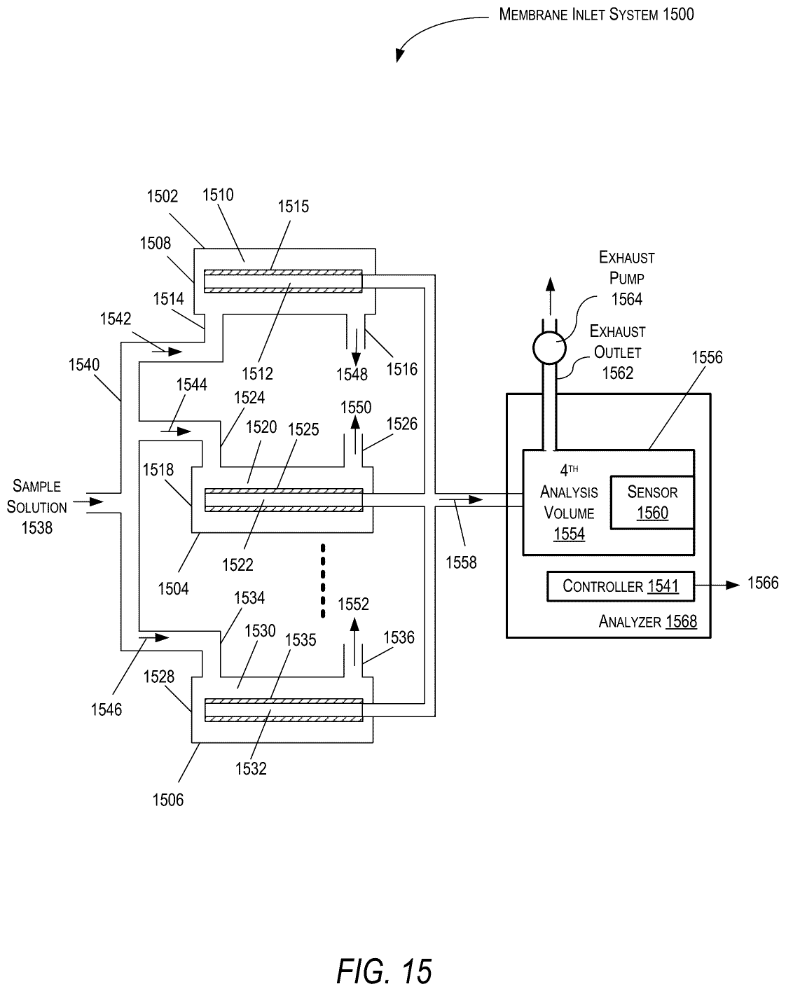

FIG. 15 is a system block diagram of a membrane inlet system 1500 utilizing multiple interconnected membrane inlets 1502, 1054, and 1506 connected in parallel in accordance with the present disclosure.

In this example, there may be any number of membrane inlets but for the ease of illustration only three membrane inlets 1502, 1504, and 1506 are shown. For purposes of ease of illustration, only sample volumes, analysis volumes, inlets, outlets, membranes, and channels from the analysis volumes are shown. Specifically, the membrane inlet system 1500 includes: the first membrane inlet 1502 including a first housing 1508 having a first sample volume 1510, a first analysis volume 1512, first inlet 1514, a first long membrane 1515, and a first outlet 1516; the second membrane inlet 1504 including a second housing 1518 having a second sample volume 1520, a second analysis volume 1522, second inlet 1524, a second long membrane 1525 and a second outlet 1526; and third membrane inlet 1506 including a second housing 1528 having a second sample volume 1530, a second analysis volume 1532, second inlet 1534, a second long membrane 1535 and a second outlet 1536.

The membrane inlet system 1500 is configured to degas a plurality of analytes within a sample solution 1538 that is injected in parallel into the three membrane inlets 1502, 1504, and 1506 via an injection path 1540 that is fluidically connected to the first inlet 1514, second inlet 1524, and third inlet 1534. Similar to previous descriptions, the first long membrane 1515 separates the first sample volume 1510 from the first analysis volume 1512, the second long membrane 1525 separates the second sample volume 1520 from the second analysis volume 1522, and the third long membrane 1535 separates the third sample volume 1530 from the third analysis volume 1532.

In an example of operation, a controller 1541 the membrane inlet system 1500 performs a method that includes producing a first constant flow 1542 of the sample solution 1538 through the first sample volume 1510 and over a first surface of the first long membrane 1515 and producing a second constant flow 1544 of the sample solution 1538 through the second sample volume 1520 and over a second surface of the second long membrane 1525. The method may also include producing a third constant flow 1546 of the sample solution 1538 through the third sample volume 1530 and over a second surface of the third long membrane 1535. The method also includes permeating a first sub-plurality of analytes from the sample solution 1538 in the first sample volume 1510 into the first analysis volume 1512 until the sample solution 1538 in the first sample volume 1510 has been approximately completely degassed prior to exiting an first outlet 1516 as first exhaust 1548 of the first sample volume 1510 and permeating the second sub-plurality of analytes from the sample solution 1538 in the second sample volume 1520 into the second analysis volume 1522 until the sample solution 1538 in the second sample volume 1520 has been approximately completely degassed prior to exiting an second outlet 1526 as second exhaust 1550 of the second sample volume 1520. The method may also include permeating the third sub-plurality of analytes from the sample solution 1538 in the third sample volume 1530 into the third analysis volume 1532 until the sample solution 1538 in the third sample volume 1530 has been approximately completely degassed prior to exiting a third outlet 1536 as second exhaust 1552 of the second sample volume 1530. The method then evacuates the first analysis volume 1512 and second analysis volume 1522, and third analysis volume 1532 into the fourth analysis volume 1554 of a fourth housing 1556 where the combined permeates 1558 are evacuated into the fourth analysis volume 1554 for measurement by the sensor 1560. The method then produces a first measurement signal, with the sensor 1560, corresponding to a first concentration a first analyte through the first long membrane 1515, second long membrane 1525, and third membrane 1535 into the first analysis volume 1512, second analysis volume 1525, and third analysis volume 1535 and a second measurement signal, with the sensor 1560, corresponding to a second concentration of a second analyte through the first long membrane 1515, second long membrane 1525, and third membrane 1535 into the first analysis volume 1512, second analysis volume 1525, and third analysis volume 1535. The combined permeates 1558 are evacuated out of the fourth analysis volume 1554 via an exhaust outlet 1562 and exhaust pump 1564. The controller 1541 then determines a ratiometric measurement 1566 for the first analyte and the second analyte based on the first measurement signal and the second measurement signal. In this example, the fourth housing 1556 and controller 1541 may be part of an analyzer 1568.

It will be understood that various aspects or details of the disclosure may be changed without departing from the scope of the disclosure. It is not exhaustive and does not limit the claimed disclosures to the precise form disclosed. Furthermore, the foregoing description is for the purpose of illustration only, and not for the purpose of limitation. Modifications and variations are possible in light of the above description or may be acquired from practicing the disclosure. The claims and their equivalents define the scope of the disclosure. Moreover, although the techniques have been described in language specific to structural features and/or methodological acts, it is to be understood that the appended claims are not necessarily limited to the features or acts described. Rather, the features and acts are described as an example implementations of such techniques.

Conditional language such as, among others, “can,” “could,” “might” or “may,” unless specifically stated otherwise, are understood within the context to present that certain examples include, while other examples do not include, certain features, elements and/or steps. Thus, such conditional language is not generally intended to imply that certain features, elements and/or steps are in any way required for one or more examples or that one or more examples necessarily include logic for deciding, with or without user input or prompting, whether certain features, elements and/or steps are included or are to be performed in any particular example. Conjunctive language such as the phrase “at least one of X, Y or Z,” unless specifically stated otherwise, is to be understood to present that an item, term, etc. may be either X, Y, or Z, or a combination thereof.

Furthermore, the description of the different examples of implementations has been presented for purposes of illustration and description, and is not intended to be exhaustive or limited to the examples in the form disclosed. Many modifications and variations will be apparent to those of ordinary skill in the art. Further, different examples of implementations may provide different features as compared to other desirable examples. The example, or examples, selected are chosen and described in order to best explain the principles of the examples, the practical application, and to enable others of ordinary skill in the art to understand the disclosure for various examples with various modifications as are suited to the particular use contemplated.

It will also be understood that various aspects or details of the invention may be changed without departing from the scope of the invention. It is not exhaustive and does not limit the claimed inventions to the precise form disclosed. Furthermore, the foregoing description is for the purpose of illustration only, and not for the purpose of limitation. Modifications and variations are possible in light of the above description or may be acquired from practicing the invention. The claims and their equivalents define the scope of the invention.

The description of the different examples of implementations has been presented for purposes of illustration and description, and is not intended to be exhaustive or limited to the examples in the form disclosed. Many modifications and variations will be apparent to those of ordinary skill in the art. Further, different examples of implementations may provide different features as compared to other desirable examples. The example, or examples, selected are chosen and described in order to best explain the principles of the examples, the practical application, and to enable others of ordinary skill in the art to understand the disclosure for various examples with various modifications as are suited to the particular use contemplated.

What is claimed is:

1. A method for chemical analysis with continuous flow sample degassing of a plurality of analytes within a sample solution utilizing a membrane inlet having a housing, a long membrane within the housing, and a sensor, wherein the housing has a sample volume and an analysis volume physically separated by the long membrane, the method comprising:
producing a constant flow of the sample solution through the sample volume and over a surface of the long membrane;
permeating the plurality of analytes from the sample solution in the sample volume into the analysis volume until the sample solution in the sample volume has been approximately completely degassed prior to exiting an outlet of the sample volume;
evacuating the analysis volume;
producing a first measurement signal, with the sensor, corresponding to a first concentration a first analyte through the long membrane into the analysis volume;
producing a second measurement signal, with the sensor, corresponding to a second concentration of a second analyte through the long membrane into the analysis volume; and
determining a ratiometric measurement for the first analyte and the second analyte based on the first measurement signal and the second measurement signal.

2. The method of claim 1, further including evacuating the permeated plurality of analytes from the analysis volume via an exhaust outlet.

3. The method of claim 1, further including controlling a rate of the flow of the sample solution through the sample volume.

4. The method of claim 1, wherein approximately completely degassed is partially degassed.

5. The method of claim 4, wherein permeating the plurality of analytes includes permeating the first analyte through the long membrane to produce the first concentration of the first analyte in the analysis volume.

6. The method of claim 5, wherein permeating the plurality of analytes further includes permeating the second analyte through the long membrane to produce the second concentration of the second analyte in the analysis volume.

7. The method of claim 1, wherein approximately completely degassed is completely degassed.

8. The method of claim 7, wherein permeating the plurality of analytes includes permeating the first analyte through the long membrane to produce the first concentration of the first analyte in the analysis volume.

9. The method of claim 8, wherein permeating the plurality of analytes further includes permeating the second analyte through the long membrane to produce the second concentration of the second analyte in the analysis volume.

10. The method of claim 1,
wherein
the plurality of analytes includes two chemical similar analytes that have different diffusion characteristics,
permeating the plurality of analytes includes
permeating the first analyte through the long membrane until the first analyte in the sample solution, in the sample volume, has been approximately completely degassed to produce the first concentration of the first analyte in the analysis volume, and
permeating the second analyte through the long membrane until the second analyte in the sample solution, in the sample volume, has been approximately completely degassed to produce the second concentration of the second analyte in the analysis volume, and
wherein the first analyte is approximately completely degassed faster than the second analytes is approximately completely degassed.

11. The method of claim 10, wherein the second analyte is an isotope of the first analyte.

12. The method of claim 11, wherein approximately completely degassed is completely degassed.

13. The method of claim 11, wherein approximately completely degassed is completely degassed.

14. The method of claim 1, wherein the sample volume is a first sample volume, the analysis volume is a first analysis volume, and the housing is a first housing, the method further comprises:
injecting the ejected sample solution from the outlet of the first sample volume into a second sample volume of a second housing, wherein
the second housing includes the second sample volume, a second long membrane, and a second analysis volume separated from the second sample volume by the second long membrane,
the second analysis volume is fluidically connected to the first analysis volume, and
the ejected approximately completely degassed sample solution is partially degassed;
evacuating the second analysis volume; and
permeating the plurality of analytes from the sample solution in the second sample volume into the second analysis volume until the sample solution in the second sample volume has been further degassed prior to exiting a second outlet of the second sample volume.

15. A method for chemical analysis with continuous flow sample degassing of a plurality of analytes within a first sample solution utilizing a first membrane inlet having a first housing, a first long membrane within the first housing, a second sample solution utilizing a second membrane inlet having a second housing, a second long membrane within the second housing, and a sensor, wherein the first housing has the first sample volume and a first analysis volume physically separated by the first long membrane, and the second housing has the second sample volume and a second analysis volume physically separated by the second long membrane, wherein the first housing and second housing are fluidically connected in parallel, the method comprising:
producing a first constant flow of the sample solution through the first sample volume and over a first surface of the first long membrane;
producing a second constant flow of the sample solution through the second sample volume and over a second surface of the second long membrane;
permeating a first sub-plurality of analytes from the sample solution in the first sample volume into the first analysis volume until the sample solution in the first sample volume has been approximately completely degassed prior to exiting an first outlet of the first sample volume;
permeating the second sub-plurality of analytes from the sample solution in the second sample volume into the second analysis volume until the sample solution in the second sample volume has been approximately completely degassed prior to exiting an second outlet of the second sample volume;
evacuating the first analysis volume and second analysis volume;
producing a first measurement signal, with the sensor, corresponding to a first concentration a first analyte through the first long membrane and second long membrane into the first analysis volume and second analysis volume;
producing a second measurement signal, with the sensor, corresponding to a second concentration of a second analyte through the first long membrane and second long membrane into the first analysis volume and second analysis volume; and
determining a ratiometric measurement for the first analyte and the second analyte based on the first measurement signal and the second measurement signal.

16. A membrane inlet for chemical analysis with continuous flow sample degassing of at least two analytes within a sample solution, the membrane inlet comprising:
a housing having a sample volume and an analysis volume, wherein the housing is configured to receive a flow of the sample solution through the sample volume;
a long membrane within the housing that physically separates the sample volume from the analysis volume, wherein the long membrane is configured to permeate the at least two analytes from the sample solution into the analysis volume;
a sensor configured to measure a concentration for each of the analytes in the analysis volume; and
a controller in signal communication with the sensor, wherein the controller includes
a memory,
a machine-readable medium having executable instructions, and
at least one processor in signal communication with the machine-readable medium, the at least one processor configured to perform operations based on the executable instructions that include:
producing a constant flow of the sample solution through the sample volume and over a surface of the long membrane;
evacuating the analysis volume;
permeating the at least two analytes from the sample solution in the sample volume into the analysis volume until the sample solution in the sample volume has been approximately completely degassed prior to exiting an outlet of the sample volume;
producing a first measurement signal, with the sensor, corresponding to a first concentration a first analyte through the long membrane into the analysis volume;
producing a second measurement signal, with the sensor, corresponding to a second concentration of a second analyte through the long membrane into the analysis volume; and
determining a ratiometric measurement for the first analyte and the second analyte based on the concentration of first analyte and the concentration of the second analyte.

17. The membrane inlet of claim 16, further including a pump for evacuating the permeated at least two analytes from the analysis volume via an exhaust outlet.

18. The membrane inlet of claim 16, wherein the controller is configured to control a rate of the flow of the sample solution through the sample volume based on chemical properties of the at least two analytes.

19. The membrane inlet of claim 16, wherein the long membrane has a length that is based on chemical properties of the at least two analytes so as to approximately completely degassed the at least two analytes.

20. The membrane inlet of claim 16, further including
a coupler having three fluidic ports,
an inner tube within the coupler,
wherein
the coupler is fluidically connected to the sample volume, analysis volume, and the outlet, and
the inner tube is fluidically connected to the analysis volume creating a channel through the coupler that is fluidically isolated from the outlet.

21. A membrane inlet for chemical analysis with continuous flow sample degassing of at least two analytes within a sample solution, the membrane inlet comprising:
a first housing having a first sample volume, a first analysis volume, and a first outlet fluidically connected to the first sample volume, wherein the first housing is configured to receive a flow of the sample solution through the first sample volume and out through the first outlet;
a first long membrane within the first housing that physically separates the first sample volume from the first analysis volume, wherein the first long membrane is configured to permeate the at least two analytes from the sample solution into the first analysis volume;
a second housing having a second sample volume, a second analysis volume, and a second outlet, wherein the second housing is configured to receive the flow of the sample solution through the second sample volume, wherein the second analysis volume is fluidically connected to the first analysis volume;
a second long membrane within the second housing that physically separates the second sample volume from the second analysis volume, wherein the second long membrane is configured to permeate the at least two analytes from the sample solution into the second analysis volume;
a sensor configured to measure a concentration for each of the analytes in the second analysis volume; and
a controller in signal communication with the sensor, wherein the controller includes
a memory,
a machine-readable medium having executable instructions, and
at least one processor in signal communication with the machine-readable medium, the at least one processor configured to perform operations based on the executable instructions that include:
producing a constant flow of the sample solution through the first sample volume and over a first surface of the first long membrane and out the first outlet;
evacuating the first analysis volume into the second analysis volume;
permeating the at least two analytes from the sample solution in the first sample volume into the first analysis volume until the sample solution in the first sample volume has been partially degassed prior to exiting the first outlet of the first sample volume;
injecting the constant flow of the sample solution from the first outlet into and through the second sample volume and over a second surface of the second long membrane;
permeating the at least two analytes from the sample solution in the second sample volume into the second analysis volume until the sample solution in the second sample volume has been approximately completely degassed prior to exiting the second outlet of the second sample volume;
producing a first measurement signal, with the sensor, corresponding to a first concentration a first analyte through the first long membrane into the first analysis volume and the second long membrane into the second analysis volume;
producing a second measurement signal, with the sensor, corresponding to a second concentration of a second analyte through the first long membrane into the first analysis volume and the second long membrane into the second analysis volume; and
determining a ratiometric measurement for the first analyte and the second analyte based on the first measurement signal and the second measurement signal.

22. The membrane inlet of claim 21, further including
a coupler having three fluidic ports,
an inner tube within the coupler,
wherein
the coupler is fluidically connected to the first sample volume, first analysis volume, and the first outlet, and
the inner tube is fluidically connected to the analysis volume creating a channel through the coupler that is fluidically isolated from the first outlet.
